(19) United States
(12) Patent Application Publication
(10) Pub. No.: US 2009/0028876 A1
(43) Pub. Date: Jan. 29, 2009

(54) METHODS OF MODIFYING CD11C+ DENDRITIC CELL DEVELOPMENT TO FORM OSTEOCLASTS FUNCTIONAL IN THE BONE ENVIRONMENT

(75) Inventor: Yen-Tung A. Teng, Rochester, NY (US)

Correspondence Address: NIXON PEABODY LLP - PATENT GROUP, 1100 CLINTON SQUARE, ROCHESTER, NY, 14604, US

(73) Assignee: UNIVERSITY OF ROCHESTER, Rochester, NY (US)

(21) Appl. No.: 11/814,515

(22) PCT Filed: Jan. 24, 2006
(86) PCT No.: PCT/US2006/002397

§ 371 Date: Apr. 16, 2008

Related U.S. Application Data
(60) Provisional application No. 60/646,941, filed on Jan. 24, 2005.

(51) Int. Cl. A61K 39/395; C12N 5/00; C12N 5/08; C12Q 1/02; A61P 19/08; C12Q 1/68; C12N 5/06; A61K 35/12

(52) U.S. Cl. 424/172.1; 435/377; 435/372; 435/352; 435/325; 424/93.7; 424/93.21; 435/29; 435/6

(57) ABSTRACT

An ex vivo method of producing osteoclasts is described that includes providing isolated CD 11 C⁺ dendritic cells and culturing the CD 11 C⁺ dendritic cells in culture medium under conditions effective to produce osteoclasts. Also disclosed are methods of up-regulating or down-regulating bone resorption by manipulating the osteoclastogenesis of CD1Ic⁺ dendritic cells either in vivo or in vitro. Methods of treating an inflammatory bone disease or a metabolic bone disorder in a subject, and screening assays to identify compounds or genes that affect myeloid osteoclastogenesis are also described.

This application claims the benefit of U.S. Provisional Patent Application Ser. No. 60/646,941, filed Jan. 24, 2005, which is hereby incorporated by reference in its entirety.

The present invention was made in part with funding received from the National Institutes of Health under grant numbers DE14473 and DE15786. The U.S. government may retain certain rights in this invention.

### BACKGROUND OF THE INVENTION

Inflammatory bone disorders, such as rheumatoid arthritis (RA), periodontal disease (PD) and osteomyelitis manifest imbalanced remodeling processes resulting in irreversible bone destruction, leading to morbidity, perturbation of life quality and even potentially life threatening conditions (Theill et al., “T cell, Bone Loss and Mammalian Evolution,” Annu. Rev. Immunol. 20: 795-823 (2002)). Bone remodeling is a tightly regulated process by a number of osteogenic cytokines, growth factors, and hormones that exert their effects on bone cells, namely osteoblasts (OB) and osteoclasts (OC). OB and OC function to control bone synthesis and resorption, respectively. OC are derived from the monocytes/macrophages (Mo/MQ) lineage in the presence of receptor activator of NF-κB ligand (RANKL; Theill et al., “T cell, Bone Loss and Mammalian Evolution,” Annu. Rev. Immunol. 20:795-823 (2002); Lacey et al., “Osteoprotegerin Ligand is a Cytokine that Regulates Osteoclast Differentiation and Activation,” Cell 93:165-176 (1998); Yasuda, et al., “Osteoclast Differentiation Factor is a Ligand for Osteoprtegerin/Osteoclastogenesis-Inhibitory Factor and is Identical to TRANCE/RANKL,” Proc. Natl. Acad. Sci. USA 95:3597-3602 (1998)). Functional OC are multinucleated giant cells that express tartrate resistant acid phosphatase (TRAP), calcitonin receptor (CT-R), cathepsin k, integrins αᵥβ₃, and are capable of resorbing bone (Teitelbaum, et al., “Genetic Regulation of Osteoclast Development & Function” Nat. Rev. Genet. 8: 638-649 (2003)).

RANKL induces osteoclastogenesis in the presence of macrophage colony stimulating factor (M-CSF or Csf-1) (Simonet et al., “Osteoprotegerin: A Novel Secreted Protein Involved in the Regulation of Bone Density,” Cell 89:309-319 (1997)), where M-CSF signals via c-fins up-regulate RANK expression on OC precursors, promoting their survival and differentiation (Biskobing et al., “Characterization of M-CSF-Induced Proliferation and Subsequent Osteoclast Formation in Murine Marrow Culture,” J. Bone Miner. Res. 10:1025-1032 (1995); Tanaka et al., “Macrophage Colony Stimulating Factor is Indispensable for Both Proliferation and Differentiation of Osteoclast Progenitors,” J. Clin. Invest. 91:257-263 (1993)). RANK, the receptor of RANKL, and its antagonist osteoprotegerin (OPG) have been shown to be the key regulators of bone remodeling and are directly involved in the differentiation, activation and survival of OC and OC precursors (Theill et al., “T cell, Bone Loss and Mammalian Evolution,”, Annu. Rev. Immunol. 20:795-823 (2002); Lacey et al., “Osteoprotegerin Ligand is a Cytokine that Regulates Osteoclast Differentiation and Activation,” Cell 93:165-176 (1998); Yasuda et al., “Osteoclast Differentiation Factor is a Ligand for Osteoprtegerin/Osteoclastogenesis-Inhibitory Factor and is Identical to TRANCE/RANKL,” Proc. Natl. Acad. Sci. USA 95:3597-3602 (1998); Li et al., “RANK is the Intrinsic Hematopoietic Cell Surface Receptor that Controls Osteoclastogenesis and Regulation of Bone Mass and Calcium Metabolism,” Proc. Natl. Acad. Sci. USA 97:1566-1571 (2000)). In addition, RANKL/RANK signaling enhances dendritic cell (DC) survival and is indispensable for lymph node organogenesis (Wong et al., “TRANCE (Tumor Necrosis Factor [TNF]-Related Activation-Induced Cytokine), a New TNF Family Member Predominantly Expressed in T Cells, is a Dendritic Cell-Specific Survival Factor,” J. Exp. Med. 186:2075-2080 (1997); Anderson et al., “A Homologue of the TNF Receptor and its Ligand Enhance T-Cell Growth and Dendritic-Cell Function,” Nature 390:175-179 (1997); Kong et al., “OPGL is a Key Regulator of Osteoclastogenesis, Lymphocyte Development and Lymph-Node Organogenesis,” Nature 397:315-323 (1999)). Genetic mutations of RANKL, RANK and M-CSF demonstrate similar defective phenotypes in OC development with severe osteopetrosis, suggesting that they are essential for osteoclastogenesis and bone remodeling (Kong et al., “OPGL is a Key Regulator of Osteoclastogenesis, Lymphocyte Development and Lymph-Node Organogenesis,” Nature 397:315-323 (1999); Dougall, et al., “RANK is Essential for Osteoclast and Lymph Node Development,” Genes Dev. 18:2412-24 (1999); Yoshida et al., “The Murine Mutation Osteopetrosis is in the Coding Region of the Macrophage Colony Stimulating Factor Gene,” Nature 345:442-444 (1990); Kodama et al., “Congenital Osteoclast Deficiency in Osteopetrotic (op/op) Mice is Cured by Injections of Macrophage-Colony Stimulating Factor,” J. Exp. Med. 173:269-272 (1991)). OPG transgenic mice are osteopetrotic with defective OC activity, whereas OPG deficient mice are severely osteoporotic (Theill et al., “T cell, Bone Loss and Mammalian Evolution,” Annu. Rev. Immunol. 20:795-823 (2002); Mizuno, et al., “Severe Osteoporosis in Mice Lacking Osteoclastogenesis Inhibitory Factor/Osteoprotegerin,” Biochem. Biophys. Res. Commun. 247:610-615 (1998)). Importantly, studies have shown that activated T-cells express RANKL and mediate osteoclastogenesis (Kong et al., “Activated T Cells Regulate Bone Loss and Joint Destruction in Adjuvant Arthritis Through Osteoprotegerin Ligand,” Nature 402: 304-308 (1999) Teng et al, “Functional Human T-Cell Immunity and Osteoprotegerin-Ligand (OPG-L) Control Alveolar Bone Destruction in Periodontal Infection,” J. Clin. Invest. 106:R59-R67 (2000)), and that blocking RANKL activity via OPG injections results in a significant reduction of bone loss in RA, PD, osteoporosis, cancer-related bone metastasis and type-1 diabetes-associated alveolar bone loss (Theill et al., “T cell, Bone Loss and Mammalian Evolution,” Annu. Rev. Immunol. 20: 795-823 (2002) Kong, et al., “Activated T Cells Regulate Bone Loss and Joint Destruction in Adjuvant Arthritis Through Osteoprotegerin Ligand,” Nature 402:304-308 (1999); Teng et al, “Functional Human T-Cell Immunity and Osteoprotegerin-Ligand (OPG-L) Control Alveolar Bone Destruction in Periodontal Infection,” J. Clin. Invest. 106:R59-R67 (2000); Hofbauer et al., “Clinical Implication of the Osteoprotegerin/RANKL/RANK System for Bone and Vascular Diseases,” JAMA 28: 490-495 (2004); Brown et al., “OPG, RANKL and RANK in Cancer Metastasis: Expression and Regulation,” Cancer Treat. Res. 118:149-172 (2004); Mahamed et al., “G(−) Anaerobes-Reactive CD4+ T-Cells Trigger RANKL-Mediated Enhanced Alveolar Bone Loss in Diabetic NOD Mice,” Diabetes 54:1477-1486 (2005)).

DC are bone marrow (BM)-derived leukocytes, with key innate immune functions including anti-viral and anti-microbial properties (Banchereau et al., “Immunobiology of Dendritic Cells,” Annu. Rev Immunol. 18:767-811 (2000)). Their wide distribution allows rapid access to sample the environment for antigens and danger signals. As professional antigen-presenting cells (APCs), DC capture antigens and efficiently prime naïve T-cells to initiate adaptive immune effector functions or to induce tolerance (Banchereau et al., “Immunobiology of Dendritic Cells,” Annu. Rev Immunol. 18:767-811 (2000); Ardavin et al., “Dendritic Cell: Immunobiology & Cancer Immunotherapy,” Immunity 1:17-23 (2004)). The origin and developmental pathways of DC remain controversial (Ardavin, “Origin, Precursors and Differentiation of Mouse Dendritic Cells,” Nat Rev. Immunol. 3:582-590 (2003)); however, they are currently classified into conventional, including myeloid & lymphoid, and plasmacytoid DC (Banchereau et al., “Immunobiology of Dendritic Cells,”, Annu. Rev Immunol. 18:767-811 (2000); Ardavin et al., “Dendritic Cell: Immunobiology & Cancer Immunotherapy,” Immunity 1:17-23 (2004); Ardavin, “Origin, Precursors and Differentiation of Mouse Dendritic Cells,” Nat Rev. Immunol. 3:582-590 (2003); Martinez del Hoyo et al., “Dendritic Cell Differentiation Potential of Mouse Monocytes: Monocytes Represent Immediate Precursors of CD8⁻ and CD8⁺ Splenic Dendritic Cells,” Blood 103:2668-2676 (2004); Traver et al., “Development of CD8a-Positive Dendritic Cells From a Common Myeloid Progenitor,” Science 290:2152-2154 (2000); Pullendran et al., “Sensing Pathogens and Tuning Immune Responses,” Science 293:253-256 (2001); Shortman, “Burnet Oration: Dendritic Cells: Multiple Subtypes, Multiple Origins, Multiple Functions,” Immunol. Cell Biol. 78:161-165 (2000)).

Recent studies reported that: i) murine DC could be Mo-derived (Martinez del Hoyo, et al., “Dendritic Cell Differentiation Potential of Mouse Monocytes: Monocytes Represent Immediate Precursors of CD8⁻ and CD8⁺ Splenic Dendritic Cells” Blood 103:2668-2676 (2004)), suggesting DC and OC may share common progenitors (Miyamoto et al., “Bifurcation of Osteoclasts and Dendritic Cells From Common Progenitors,” Blood 98:2544-2554 (2001)), and that Flt3⁺MQ precursors may differentiate sequentially to OC, DC and microglia (Servet-Delprat et al., “Flt3⁺ Macrophage Precursors Commit Sequentially to Osteoclasts, Dendritic Cells and Microglia,” BMC Immunol 3(1):15 (2002)), ii) immature DC “transdifferentiate” to OC in RA, suggesting that DC may directly contribute to inflammation-induced osteoclastogenesis (Rivollier et al., “Immature Dendritic Cell Transdifferentiation into Osteoclasts: A Novel Pathway Sustained by Rheumatoid Arthritis Microenvironment,” Blood 104:4029-4037 (2004)), and iii) Langerhan's cells (LC) may give rise to multinucleated TRAP⁺OC-like cells in the inflammatory LC-histiocytosis lesions (Da Costa et al., “Presence of Osteoclast-Like Multinucleated Giant Cells in the Bone and Nonostotic Lesions of Langerhan's Cell Histiocytosis” J. Exp. Med. 201(5):687-693 (2005)). Further, DC can activate naïve T-cells in the presence of microbial antigens such as Lactobacillus spp. and Helicobactor pylori (Mohamadzadeh et al., “Lactobacilli Activate Human Dendritic Cells that Skew T Cells Towards T Helper 1 Polarization,” Proc. Natl. Acad. Sci. USA 102:2880-2885 (2005); Hafsi et al., “Human Dendritic Cells Respond to Helicobacter Pylori, Promoting NK-Cell and Th1-Effector Responses in Vitro,” J Immunol. 173:1249-1257 (2004)).

DC are present in active disease sites of RA and PD where they aggregate with T-cells in the inflammatory foci (Thomas et al., “Dendritic Cells and the Pathogenesis of Rheumatoid Arthritis,” J Leukoc Biol 66:286-92 (1999); Highton et al., “Cells Expressing Dendritic Cell Markers are Present in the Rheumatoid Nodule,” J. Rheumatol. 27:339-346 (2000); Cirrincione et al., “Lamina Propria Dendritic Cells Express Activation Markers and Contact Lymphocytes in Chronic Periodontitis,” J Periodontol. 73:45-52 (2002); Teng, “The Role of Acquired Immunity and Periodontal Disease Progression,” Crit. Rev Oral Biol Med. 14:237-252 (2003)) and are thought to contribute indirectly to inflammation-induced bone loss in RA (Santiago-Schwarz et al., “Dendritic Cells (DCs) in Rheumatoid Arthritis (RA): Progenitor Cells and Soluble Factors Contained in RA Synovial Fluid Yield a Subset of Myeloid DCs that Preferentially Activate Th1 Inflammatory-Type Responses,” J Immunol. 67:1758-1768 (2001)). Despite their direct contribution to osteoclastogenesis has recently been proposed, where Rivollier et al showed that human blood Mo-derived DC can “transdifferentiate” into OC in the presence of M-CSF and RANKL in vitro (Rivollier et al., “Immature Dendritic Cell Transdifferentiation into Osteoclasts: A Novel Pathway Sustained by Rheumatoid Arthritis Microenvironment,” Blood 104:4029-4037 (2004)), it remains unclear whether DC/T-cell interactions support such development, and whether DC can directly contribute to inflammation-induced osteoclastogenesis during immune interactions and activation in the bone environment.

The present application describes the novel findings that BM-derived CD11c⁺DC, the classical myeloid DC, can indeed develop into functional OC, undergoing osteoclastogenesis when properly activated in the bone environment. Thus, the present invention overcomes the above-identified deficiencies in the art.

### SUMMARY OF THE INVENTION

A first aspect of the present invention relates to an ex vivo method of producing osteoclasts that includes the steps of: providing isolated CD11c⁺ dendritic cells; culturing the CD11c⁺ dendritic cells in culture medium under conditions effective to produce an osteoclast precursor population; and treating the osteoclast precursor population under conditions effective to induce differentiation of the osteoclast precursor population to form osteoclasts.

A second aspect of the present invention relates to osteoclasts produced according to the first aspect of the present invention.

A third aspect of the present invention relates to a method of upregulating bone resorption in a subject that includes the steps of: performing the method according to the first aspect of the present invention to form osteoclasts using CD11c⁺ dendritic cells isolated from a subject; and returning the osteoclasts to the subject, thereby upregulating bone resorption in the subject.

A fourth aspect of the present invention relates to a method of upregulating bone resorption in a subject that includes the steps of: providing CD11c⁺ dendritic cells isolated from a subject; introducing a nucleic acid molecule to the CD11c⁺ dendritic cells to form transgenic CD11c⁺ dendritic cells, wherein the nucleic acid molecule encodes a protein or polypeptide capable of promoting osteoclastogenesis of the dendritic cells; and returning the transgenic cells to the subject, thereby upregulating bone resorption in the subject. This method encompasses both return of transgenic osteoclast precursor cells and return of functional transgenic osteoclasts.

A fifth aspect of the present invention relates to a method of upregulating bone resorption in a subject that includes the steps of: providing naïve CD4⁺ T cells isolated from the subject; co-culturing the CD4⁺ T cells with a peptide or polypeptide antigen and CD11c⁺ dendritic cells under conditions effective to promote osteoclastogenesis of the CD11c⁺ dendritic cells; and returning the cultured CD4⁺ T cells to the subject, where they are in contact with CD11c⁺ dendritic cells, thereby promoting osteoclastogenesis of the CD11c⁺ dendritic cells and upregulating bone resorption in the subject.

A sixth aspect of the present invention relates to a method of downregulating bone resorption in a subject that includes the steps of: providing CD11c⁺ dendritic cells isolated from a subject; introducing a nucleic acid molecule to the CD11c⁺ dendritic cells to form transgenic CD11c⁺ dendritic cells, wherein the nucleic acid molecule expresses a protein or polypeptide or an RNA molecule capable of inhibiting osteoclastogenesis in CD11c⁺ dendritic cells; and returning the transgenic CD11c⁺ dendritic cells to the subject, thereby downregulating bone resorption in the subject.

A seventh aspect of the present invention relates to a method of inhibiting osteoclastogenesis of CD11c⁺ dendritic cells in a subject that includes the steps of: administering to a subject an effective amount of an anti-RANKL antibody, wherein said administering is effective to contact RANKL in the proximity of CD11c⁺ dendritic cells, thereby inhibiting RANKL-mediated osteoclastogenesis of the CD11c⁺ dendritic cells. This can be used to downregulate bone resorption in the subject.

An eighth aspect of the present invention relates to a method of downregulating bone resorption in a subject that includes the steps of: providing naïve CD4⁺ T cells isolated from a subject; introducing a nucleic acid molecule to the CD4⁺ T cells to form transgenic CD4⁺ T cells, wherein the nucleic acid molecule encodes a protein or polypeptide that inhibits osteoclastogenesis in CD11c⁺ dendritic cells; and returning the transgenic CD4⁺ T cells to the subject, where the T cells inhibit osteoclastogenesis of CD11c⁺ dendritic cells, thereby downregulating bone resorption in the subject.

A ninth aspect of the present invention relates to a method of treating a bone disease or disorder in a subject using any of the methods of the present invention. Exemplary diseases or disorders include, without limitation, osteoporosis, osteoarthritis, osteomyelitis, rheumatoid arthritis, periodontal disease, Pagets Disease, and osteopetrosis. This subject can be any mammal, preferably a human.

A tenth aspect of the present invention relates to a method of screening for compounds which affect osteoclastogenesis that includes the steps of: performing the method according to the first aspect of the present invention under conditions effective to produce osteoclasts; exposing the dendritic cell or osteoclast precursor to a compound; and determining the ability of the compound of affect osteoclastogenesis.

An eleventh aspect of the present invention relates to a method of identifying genes related to the production of functional osteoclasts from CD11c⁺ dendritic cells. The method includes the steps of: performing the method according to claim 1 while altering one or more conditions; determining osteoclast production and function in the culture; and screening for one or more genes associated with a change in osteoclast production or function in the culture compared with a culture in which culture conditions were not altered.

The development of functional OC has been demonstrated using CD11c⁺DC subset(s), and the OC are capable of inducing bone resorption in vitro and in vivo. The dynamic process of OC differentiation, development and activation from CD11c⁺DC through orchestrated M-CSF and RANKL signaling (and overall activation signals) illustrates the complexity of DC biology. This DC plasticity supports a link between innate immunity and osteoclastogenesis, beyond the current paradigm of osteoimmunology, and furthers our understanding of the immune interactions involved in bone remodeling under pathological conditions such as RA, PD, osteomyelitis and other inflammatory bone disorders

### BRIEF DESCRIPTION OF THE DRAWINGS

FIGS. 1A-I illustrate CD11c⁺DC development of TRAP and resorptive activities in a RANKL dependent manner during co-cultures with CD4⁺T-cells and sonicated Aa antigens in vitro. 0.5×10⁶ Balb/c BM-derived MACS-purified CD11c⁺DC were cultured with Aa (10 μg/ml) sonicated antigens with or without 0.5×10⁶ syngeneic CD4⁺T-cells or sRANKL (30 ng/ml). On day 5, cells were fixed and stained for TRAP; TRAP(+) multinucleated cells containing ≧3 nuclei were included in the quantitative analysis. FIG. 1A shows that DC express significantly higher levels of TRAP (p≦0.03) after co-culture with naïve syngeneic CD4⁺T-cells and Aa when compared to negative controls. FIG. 1B shows that TRAP expression was abolished by OPG (p=0.01) and anti-RANK Ab (p=0.01) compared to DC+T-cells+Aa, and significantly increased in DC+RANKL (30 ng/ml)+Aa (p=0.008) compared to DC+RANKL alone. FIG. 1C shows that TRAP expression was significantly reduced when DC/T cell interaction was blocked by anti-MHC-II Ab (p=0.008) or anti-TCR/CD3 Abs (p=0.02) compared to DC+T-cells+Aa. To assess OC function, cells were stripped by 1N NaOH on day 5 and the total surface area of HA resorption was quantified. FIG. 1D shows that DDOC induced significant bone resorption in T-cells+Aa co-culture compared to negative controls (p≦0.03). FIG. 1E shows that both OPG and anti-RANK Ab blocked their resorptive ability (p=0.02 & p=0.007, respectively); RANKL restored this ability in the presence of Aa stimulation (p=0.01) compared to DC+RANKL alone. FIG. 1F shows that blocking DC/T cell interaction by anti-MHC-II Ab or anti-TCR/CD3 Abs abolished resorptive pit activity (p=0.009 & p=0.01, respectively) compared to DC+T-cells+Aa. FIG. 1G shows representative TRAP and resorptive activities on day 5 on hydroxyapatite from DC+T-cells+Aa, DC+RANKL+Aa and DC+T-cells+Aa⁺OPG. Multinucleated cells, and DDOC in resorption pits on synthetic bone in DC+T+Aa. FIG. 1H illustrates representative images of actin-ring formation on day 5 (IF: 400×). FIG. 1I shows Balb/c BM-derived & MACS-purified “splenic” CD11c⁺DC expressed comparable levels of TRAP indicating similar osteoclastogenic potential. The results are shown as the total surface area (in mm²), total number of TRAP(+) cells or resorptive pits per well (mean±S.E.M.) from five sets of independent experiments using total splenocytes+ConA as the positive control. The results of splenic CD11c⁺DC resorptive pits assay mirrored the TRAP data.

FIGS. 2A-D illustrate CD11c⁺DC development into TRAP (+) cells with resorptive activity in response to protein antigens. 10⁵ Balb/c BM-derived CD11c⁺DC were co-cultured with syngeneic CD4⁺ T-cells in the presence of Aa (10 μg/ml), OMP-1 or BI (20 μg/ml). Day 5 TRAP and resorptive activities were quantified. Note that T-cells in DC+T+Aa co-cultures were purified from naïve mice, while those in DC+T+Omp-1 or BI were purified from Omp-1/CFA and BI/CFA immunized Balb/c mice, respectively. FIG. 2A shows that Aa, OMP-1 and BI induced similar levels of TRAP and resorptive activities; total surface area of TRAP⁽⁺⁾ DC (top panel) and resorptive pits (bottom panel) in Aa, OMP-1 and BI co-cultures were comparable (p>0.05). FIG. 2B shows that freshly isolated immature CD11c⁺DC were stained for CD11b, F4/80, ER-MP12 & ER-MP20 to exclude Mo/MQ contamination. FIGS. 2C-D show that the levels of TRAP and resorptive pit activities by DDOC, generated from BM with or without Mo/MQ depletion, in CD4⁺T-cell+Aa co-cultures were comparable (p≧0.5 & p≧0.4, respectively). Representative data from three independent experiments are shown.

FIGS. 3A-D illustrate activated CD11c⁺DC develop functional OC phenotype during co-cultures with CD4⁺ T-cells and Aa. FIG. 3A shows that freshly purified Balb/c BM-derived CD11c⁺DC induced proliferation of allogeneic T cells in MLR and activation of syngeneic CD4⁺T-cells as indicated by CD25 (red) and RANKL (green) expression (see dark & light arrows). Freshly purified CD11c⁺DC or collected after 3 and 5 days co-cultures with CD4⁺ T-cell+Aa with or without OPG (30 ng/ml) were stained to characterize their OC vs. DC phenotypes. FIG. 3B is a comparison of immature and activation profiles of CD11c⁺DC on day 0 & 12 hrs post co-cultures compared to DC+LPS; where sMHC-II & iMHC-II indicate surface & intracellular MHC-II levels respectively. Dot plots depict the percentage of DC expressing surface CD11c, GM-CSFR, RANK, MHC-II, CT-R and intracellular cathepsin-k on days 0, 3 & 5±OPG. FIG. 3C is a summary of cell-surface profile of CT-R(+) DDOC co-stained for CD11c, GM-CSFR, RANK and MHC-II expressions. FIG. 3D illustrates SIDIA of CD11c⁺DC (day 0) & DDOC (days 3 & 5) fluorescently stained for CT-R and RANK or CT-R and GM-CSFR shown as MFI in pixels±S.E. at the single cell level. Representative data out of three independent experiments with similar results are shown here.

FIG. 4A-D illustrate that M-CSF is not required for OC development in the present co-cultures, but is essential for the acquisition of CD11c⁺DC potential to act like OC precursors capable of further development into functional OC. 0.5×10⁶ WT BM-derived CD11c⁺DC were co-cultured with CD4⁺ T-cells+Aa (10 μg/ml) in the presence of anti-M-CSF Ab (10 μg/ml). FIG. 4A shows the total surface area of TRAP and resorptive pits formed by WT DDOC remained unchanged after the addition of anti-M-CSF Ab. 10⁵ Csf-1^{-/-}CD11c⁺DC were co-cultured with WT CD4⁺T-cells or sRANKL (30 ng/ml) and Aa with or without exogenous rmM-CSF (25 ng/ml) for 5 days using WT CD11c⁺DC co-cultures as positive control. The addition of M-CSF to Csf-1^{-/-}op/op CD11c⁺DC co-culture, or together with RANKL+Aa, significantly restored TRAP (FIG. 4B) and resorptive pit (FIG. 4C) activities by day 5 as measured by total surface area of TRAP(+) cells and resorptive pit in mm² (n=3). FIG. 4C illustrates, at the clonal level, that there was no significant difference in MFI of CT-R, GM-CSFR and RANK expressed by Csf-1^{-/-}op/op DDOC in co-cultures with M-CSF on day 5, compared to WT DDOC (p≧0.2).

FIGS. 5A-I illustrate that DDOC are capable of inducing bone resorption in vivo. 2×10⁶ CFSE- and Cy5-labelled WT CD11c⁺DC were co-cultured with CD4⁺T-cells+Aa for 2-3 days, then cells were collected and injected onto NOD/SCID mice calvarias. Controls included PBS injection and injection of 2×10⁶ viable CD4⁺T-cells or CD11c⁺DC with or without Aa (n=3 mice/group). All mice were sacrificed 4 days post-injection. The results of one of three experiments with similar results are shown here. FIGS. 5A-B shows H&E staining of the control mice receiving PBS or DC+Aa, respectively (*indicate CFSE⁽⁺⁾CD11c⁺DC injected in vivo; 100×-400× magnification). Note: Smooth calvarial bone surface with no erosion or resorption detected. Injection of control CD4⁺T-cells alone did not yield any significant bone resorption. FIG. 5C shows H&E staining of mice calvaria exhibiting bone resorption after injection of in vitro generated DDOC (100×). FIG. 5D shows serial sections of the calvarias post-injection (of FIG. 5G: 400×) showing cellular infiltration with bone resorption. FIG. 5E shows, at higher power, the bone resorptive area of FIG. 5D (600×). OC-like cells with lacunae appear on calvarial bone surface. FIG. 5F shows CFSE⁽⁺⁾CD11c⁺DC (co-cultured with Aa only) residing on “smooth” calvarial bone surface post-injection in vivo (400×). FIG. 5G shows CFSE⁽⁺⁾ DDOC residing in a large “resorptive and eroded” area on calvarial bone surface (the same proximal section of FIG. 5D: 400×). FIGS. 5H-I shows immunohistochemical staining of TRAP on DDOC in various samples and sections of the resorbed bone area in calvarias (600×).

FIGS. 6A-C illustrate the histological perturbations in arthritic joints in a mouse model at 16× and 400× magnification (FIG. 6A-B), with CD11c⁺ staining in FIG. 6C (400×). Arthritis was induced in knee joints by administration of chicken type-II collagen according to accepted procedures of Stuart et al., J. Clin. Invest. 69:673-683 (1982), which is hereby incorporated by reference in its entirety.

FIGS. 7A-D illustrate the histology in normal joints of control mice at 16× and 150× (FIGS. 7A, 7C), and CD11c⁺ staining in FIGS. 7B (100×) and 7D (400×).

### DETAILED DESCRIPTION OF THE INVENTION

The present invention relates to methods of modulating CD11c⁺DC osteoclastogenesis either ex vivo or in vivo, and use of such modulation to enhance or diminish the population of DC-derived osteoclasts for therapeutic purposes. The resulting CD11c⁺DC-derived osteoclasts are phenotypically distinct yet possess bone resorbing capability.

One aspect of the present invention relates to an ex vivo approach for promoting osteoclastogenesis of CD11c⁺DC. Isolated CD11c⁺DC are cultured in a competent culture medium to promote development of the CD11c⁺DCs into an osteoclast precursor population. Thereafter, the osteoclasts precursor population is preferably substantially purified so that the osteoclast precursor population is substantially free of CD11b⁺cells. The purified osteoclast precursor population is then treated in a manner to induce differentiation of the osteoclast precursor population to form osteoclasts.

The CD11c⁺DCs can be isolated from any tissue that is sufficiently rich in this cell population. Exemplary tissues include, without limitation, bone marrow, peripheral blood, spleen, and lymph nodes. The CD11c⁺DC are mammalian cells, and preferably though not exclusively human CD11c⁺DC. CD11c⁺DC from other mammals can also be used, including non-human primates, rodents such as mice and rats, dogs, cats, horses, cows, sheep, pigs, llama, etc.

The culture medium preferably contains macrophage colony stimulating factor and IL-4. Culturing can be carried out for about two to about seven days, and the medium should be refreshed periodically (i.e., daily). The culturing at this stage is sufficient to allow the CD11c⁺DC to form osteoclast precursor cells.

Purification can be carried out using any suitable means. Purification is preferably, but not necessarily, performed subsequent to the initial culturing step described above. A preferred approach involves use of a cell sorter, such as a magnetic cell sort with magnetic immuno-separation of the CD11c⁺ cells from CD11b⁺ cells. The resulting purified osteoclast precursor population is preferably at least about 95% pure (with regard to CD11b⁺ cells), more preferably greater than 98% pure, most preferably greater than 99% pure.

Regardless of the approach used to obtain the CD11c⁺ population of purified osteoclast precursors, this population can be treated in a manner effective to induce differentiation of the osteoclast precursors to form osteoclasts. Basically, the precursors are exposed to a peptide or polypeptide antigen, receptor activator of NF-1-κB ligand (RANKL), and either bone or a bone substitute.

The RANKL can be introduced directly into culture medium, i.e., recombinant or purified RANKL can be added, RANKL-expressing CD4 T cells can be introduced, or both.

The antigen introduced to the osteoclast precursors can be any antigen, but preferably a microbial antigen. Successful results have been obtained using, e.g., antigen of A. actinomycetemcomitans and P. gingivalis. The antigen need not be from a pathogen of the mammal from which the CD11c⁺DC were isolated.

The bone or bone substitute can be in the form of harvested or pulverized bone tissue, dentin, synthetic bone available from commercial sources (see examples), or hydroxyapatite.

The resulting osteoclasts produced in this manner are characterized by, inter alia, one or more of the following phenotype markers: CD11c⁺, CD11b⁻, TRAP⁺, and CT-R⁺. In addition, the resulting osteoclasts are capable of resorbing bone either in vitro or in vivo. The osteoclasts can be use for therapeutic treatments described hereinafter.

In other aspects of the present invention, transgenic CD11c⁺ can be prepared. Basically, a nucleic acid molecule encoding a protein or polypeptide capable of regulating osteoclastogenesis in CD11c⁺ dendritic cells is introduced to CD11c⁺ dendritic cells to form transgenic dendritic cells. The nucleic acid molecule of choice can be introduced into an expression system or vector of choice using conventional recombinant technology. Generally, this involves inserting the nucleic acid molecule into an expression system to which the molecule is heterologous (i.e., not normally present). One or more desired nucleic acid molecules may be inserted into the vector. When multiple nucleic acid molecules are inserted, the multiple nucleic acid molecules may encode the same or different nucleic acid molecules. The heterologous nucleic acid molecule is inserted into the expression system or vector in proper sense (5′→3′) orientation relative to the promoter and any other 5′ regulatory molecules, and correct reading frame, when expression of the encoded protein or polypeptide in the transgenic CD11c⁺ dendritic cells is desired. Alternatively, the nucleic acid may be inserted in the “antisense” orientation relative to the promoter and any other 5′ regulatory molecules, i.e, in a 3′→5′ prime direction, such that antisense RNA is produced when transcription of the nucleic acid molecule occurs. In each of these aspects, the vector contains the necessary elements for the transcription and translation of the inserted protein or polypeptide-coding sequences. The orientation of the nucleic acid molecule will be dependent on whether the regulation of osteoclastogenesis in the dendritic cells is intended to be a downregulation or an upregulation. Where upregulation is intended, a suitable nucleic acid molecule is inserted in the sense orientation to allow expression of a protein or polypeptide capable of stimulating osteoclastogenesis in the transgenic CD11c⁺ dendritic cells to occur. When the intended regulation is a downregulation (which is meant to include also complete abrogation of osteoclastogenesis), a suitable nucleic acid molecule may be inserted in the antisense orientation. Nucleic acid molecules suitable for this aspect of the present invention are bone modulating peptides, proteins, chemokines, cytokines, growth hormones, or monoclonal antibodies, including, without limitation: osteoprotegerin, tumor necrosis factor-β, and transforming growth factor-β, GM-CSF, anti-RANKL antibodies, and antibodies to any other bone-modulating factors. The term “bone modulating factor” as used herein refers to any factor that influences bone and mineral metabolism. This is meant to include those factors that up-regulate and down-regulation bone and mineral metabolism, which may result in up- or down-regulation of bone resorption and bone generation (and regeneration). Such factors include those capable of inhibiting or promoting osteoclastogenesis in CD11c⁺ dendritic cells, in vivo or ex vivo.

Antisense nucleic acids are DNA or RNA molecules or oligoribonucleotides or oligodeoxyribonucleotides that are complementary to at least a portion of a specific mRNA molecule (Weintraub, Scientific American 262:40 (1990), which is hereby incorporated by reference in its entirety). In the cell, the antisense nucleic acids are transcribed and hybridize to a target nucleic acid. The specific hybridization of an antisense nucleic acid molecule with its target nucleic acid interferes with the normal function of the target nucleic acid. The functions of DNA to be interfered with include replication and transcription. The functions of RNA to be interfered with include all vital functions, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is the regulation of the protein expression.

In the aspect of the present invention in which down-regulation of osteoclastogenesis is desired, the method of interfering with endogenous protein expression may involve an RNA-based form of gene-silencing known as RNA-interference (RNAi) (also known more recently as siRNA for short, interfering RNAs). RNAi is a form of post-transcriptional gene silencing (PTGS). PTGS is the silencing of an endogenous gene caused by the introduction of a homologous double-stranded RNA (dsRNA), transgene, or virus. In PTGS, the transcript of the silenced gene is synthesized, but does not accumulate because it is degraded. RNAi is a specific from of PTGS, in which the gene silencing is induced by the direct introduction of dsRNA. Numerous reports have been published on critical advances in the understanding of the biochemistry and genetics of both gene silencing and RNAi (Matzke et al., “RNA-Based Silencing Strategies in Plants,” Curr Opin Genet Dev 11(2):221-227 (2001), Hammond et al., “Post-Transcriptional Gene Silencing by Double-Stranded RNA,” Nature Rev Gen 2:110-119 (Abstract) (2001); Hamilton et al., “A Species of Small Antisense RNA in Posttranscriptional Gene Silencing in Plants,” Science 286:950-952 (Abstract) (1999); Hammond et al., “An RNA-Directed Nuclease Mediates Post-Transcriptional Gene Silencing in Drosophila Cells,” Nature 404:293-298 (2000); Hutvagner et al., “RNAi: Nature Abhors a Double-Strand,” Curr Opin Genetics & Development 12:225-232 (2002), which are hereby incorporated by reference in their entirety). In iRNA, the introduction of double stranded RNA (dsRNA) into animal or plant cells leads to the destruction of the endogenous, homologous mRNA, phenocopying a null mutant for that specific gene. In siRNA, the dsRNA is processed to short interfering molecules of 21-, 22- or 23-nucleotide RNAs (siRNA), which are also called “guide RAs,” (Hammond et al., “Post-Transcriptional Gene Silencing by Double-Stranded RNA,” Nature Rev Gen 2:110-119 (Abstract) (2001); Sharp, P. A., “RNA Interference-2001,” Genes Dev 15:485-490 (2001); Hutvagner et al., “RNAi: Nature Abhors a Double-Strand,” Curr Opin Genetics & Development 12:225-232 (2002), which are hereby incorporated by reference in their entirety) in vivo by the Dicer enzyme, a member of the RNAse III-family of dsRNA-specific ribonucleases (Hutvagner et al., “RNAi: Nature Abhors a Double-Strand,” Curr Opin Genetics & Development 12:225-232 (2002); Bernstein et al., “Role for a Bidentate Ribonuclease in the Initiation Step of RNA Interference,” Nature 409:363-366 (2001); Tuschl, T., “RNA Interference and Small Interfering RNAs,” Chembiochem 2:239-245 (2001); Zamore et al., “RNAi: Double Stranded RNA Directs the ATP-Dependent Cleavage of mRNA at 21 to 23 Nucleotide Intervals,” Cell 101:25-3 (2000); U.S. Pat. No. 6,737,512 to Wu et al., which are hereby incorporated by reference in their entirety). Successive cleavage events degrade the RNA to 19-21 bp duplexes, each with 2-nucleotide 3′ overhangs (Hutvagner et al., “RNAi: Nature Abhors a Double-Strand,” Curr Opin Genetics & Development 12:225-232 (2002); Bernstein et al., “Role for a Bidentate Ribonuclease in the Initiation Step of RNA Interference,” Nature 409:363-366 (2001), which are hereby incorporated by reference in their entirety). The siRNAs are incorporated into an effector known as the RNA-induced silencing complex (RISC), which targets the homologous endogenous transcript by base pairing interactions and cleaves the mRNA approximately 12 nucleotides form the 3′ terminus of the siRNA (Hammond et al., “Post-Transcriptional Gene Silencing by Double-Stranded RNA,” Nature Rev Gen 2:110-119 (Abstract) (2001); Sharp, P. A., “RNA Interference-2001,” Genes Dev 15:485-490 (2001); Hutvagner et al., “RNAi: Nature Abhors a Double-Strand,” Curr Opin Genetics & Development 12:225-232 (2002); Nykanen et al., “ATP Requirements and Small Interfering RNA Structure in the RNA Interference Pathway,” Cell 107:309-321 (2001), which are hereby incorporated by reference in their entirety).

There are several methods for preparing siRNA, including chemical synthesis, in vitro transcription, siRNA expression vectors, and PCR expression cassettes. In one aspect of the present invention, dsRNA for the nucleic acid molecule of the present invention can be generated by transcription in vivo. This involves modifying the nucleic acid molecule of the present invention for the production of dsRNA, inserting the modified nucleic acid molecule into a suitable expression vector having the appropriate 5′ and 3′ regulatory nucleotide sequences operably linked for transcription and translation, as described above, and introducing the expression vector having the modified nucleic acid molecule into a suitable host or subject. Using siRNA for gene silencing is a rapidly evolving tool in molecular biology, and guidelines are available in the literature for designing highly effective siRNA targets and making antisense nucleic acid constructs for inhibiting endogenous protein (U.S. Pat. No. 6,737,512 to Wu et al.; Brown et al., “RNA Interference in Mammalian Cell Culture: Design, Execution, and Analysis of the siRNA Effect,” Ambion TechNotes 9(1):3-5 (2002); Sui et al., “A DNA Vector-Based RNAi Technology to Suppress Gene Expression in Mammalian Cells,” Proc Natl Acad Sci USA 99(8):5515-5520 (2002); Yu et al., “RNA Interference by Expression of Short-Interfering RNAs and Hairpin RNAs in Mammalian Cells,” Proc Natl Acad Sci USA 99(9):6047-6052 (2002); Paul et al., “Effective Expression of Small Interfering RNA in Human Cells,” Nature Biotechnology 20:505-508 (2002); Brummelkamp et al., “A System for Stable Expression of Short Interfering RNAs in Mammalian Cells,” Science 296:550-553 (2002), which are hereby incorporated by reference in their entirety). There are also commercially available sources for custom-made siRNAs.

The preparation of the nucleic acid constructs of the present invention including a nucleic acid molecule suitable to regulate osteoclastogenesis in CD11c⁺ dendritic cells is carried out using methods well known in the art. U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture. Other vectors are also suitable.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK +/− or KS +/− (see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Human gene therapy is an approach to treating human disease that is based on the modification of gene expression in cells of the patient. Eukaryotic viruses have been employed as vehicles for somatic gene therapy. Among the viral vectors that have been cited frequently in gene therapy research are adenoviruses (U.S. Pat. No. 6,203,975 to Wilson). Several viral systems including murine retrovirus, adenovirus, parvovirus (adeno-associated virus), vaccinia virus, and herpes virus have been developed as therapeutic gene transfer vectors (for review see, Nienhuis et al., Hematology, Vol. 16: Viruses and Bone Marrow, N. S. Young (ed.), pp. 353-414 (1993), which is hereby incorporated by reference in its entirety). Viral vectors provide a more efficient means of transferring genes into cells as compared to other techniques such as calcium phosphate or DEAE-dextran-mediated transfection, electroporation, or microinjection. It is believed that the efficiency of viral transfer is due to the fact that the transfer of DNA is a receptor-mediated process (i.e., the virus binds to a specific receptor protein on the surface of the cell to be infected).

Once a suitable expression vector is selected, the desired nucleic acid sequences are cloned into the vector using standard cloning procedures in the art, as described by Sambrook et al., Molecular Cloning: A Laboratory Manual, Cold Springs Laboratory, Cold Springs Harbor, N.Y. (1989), or U.S. Pat. No. 4,237,224 to Cohen and Boyer, which are hereby incorporated by reference in their entirety. The vector is then introduced to a suitable host.

A variety of host-vector systems may be utilized to express the recombinant protein or polypeptide inserted into a vector as described above. Primarily, the vector system must be compatible with the host used. Host-vector systems include, without limitation, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used to carry out this and other aspects of the present invention.

Different genetic signals and processing events control many levels of gene expression (e.g., DNA transcription and messenger RNA (“mRNA”) translation). Transcription of DNA is dependent upon the presence of a promoter, which is a DNA sequence that directs the binding of RNA polymerase, and thereby promotes mRNA synthesis. The DNA sequences of eukaryotic promoters differ from those of prokaryotic promoters. Furthermore, eukaryotic promoters and accompanying genetic signals may not be recognized in, or may not function in, a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, on pF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Common promoters suitable for directing expression in mammalian cells include, without limitation, SV40, MMTV, metallothionein-1, adenovirus E1a, CMV, immediate early, immunoglobulin heavy chain promoter and enhancer, and RSV-LTR.

When multiple nucleic acid molecules are inserted, the multiple nucleic acid molecules may all be placed under a single 5′ regulatory region and a single 3′ regulatory region, where the regulatory regions are of sufficient strength to transcribe and/or express the nucleic acid molecules as desired.

Specific initiation signals are also required for efficient gene transcription and translation in prokaryotic cells. These transcription and translation initiation signals may vary in “strength” as measured by the quantity of gene specific messenger RNA and protein synthesized, respectively. The nucleic acid expression vector, which contains a promoter, may also contain any combination of various “strong” transcription and/or translation initiation signals. For instance, efficient translation in E. coli requires a Shine-Dalgarno (“SD”) sequence about 7-9 bases 5′ to the initiation codon (ATG) to provide a ribosome binding site. Thus, any SD-ATG combination that can be utilized by host ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used. Depending on the vector system and host utilized, any number of suitable transcription and/or translation elements, including constitutive, inducible, and repressible promoters, as well as minimal 5′ promoter elements, enhancers or leader sequences may be used.

Typically, when a recombinant host is produced, an antibiotic or other compound useful for selective growth of the transgenic cells only is added as a supplement to the media. The compound to be used will be dictated by the selectable marker element present in the plasmid with which the host was transformed. Suitable genes are those which confer resistance to gentamycin, G418, hygromycin, streptomycin, spectinomycin, tetracycline, chloramphenicol, and the like. Similarly, “reporter genes,” which encode enzymes providing for production of an identifiable compound identifiable, or other markers which indicate relevant information regarding the outcome of gene delivery, are suitable. For example, various luminescent or phosphorescent reporter genes are also appropriate, such that the presence of the heterologous gene may be ascertained visually.

An example of a marker suitable for the present invention is the green fluorescent protein (GFP) gene. The isolated nucleic acid molecule encoding a green fluorescent protein can be deoxyribonucleic acid (DNA) or ribonucleic acid (RNA, including messenger RNA or mRNA), genomic or recombinant, biologically isolated or synthetic. The DNA molecule can be a cDNA molecule, which is a DNA copy of a messenger RNA (mRNA) encoding the GFP. In one embodiment, the GFP can be from Aequorea victoria (Prasher et al., “Primary Structure of the Aequorea Victoria Green-Fluorescent Protein,” Gene 111(2):229-233 (1992); U.S. Pat. No. 5,491,084 to Chalfie et al., which are hereby incorporated by reference in their entirety). A plasmid encoding the GFP of Aequorea victoria is available from the ATCC as Accession No. 75547. Mutated forms of GFP that emit more strongly than the native protein, as well as forms of GFP amenable to stable translation in higher vertebrates, are commercially available from Clontech Laboratories, Inc. (Palo Alto, Calif.) and can be used for the same purpose. The plasmid designated pTα1-GFPh (ATCC Accession No. 98299, which is hereby incorporated by reference in its entirety) includes a humanized form of GFP. Indeed, any nucleic acid molecule encoding a fluorescent form of GFP can be used in accordance with the subject invention. Standard techniques are then used to place the nucleic acid molecule encoding GFP under the control of the chosen cell specific promoter.

The selection marker employed will depend on the target species and/or host or packaging cell lines compatible with a chosen vector.

A nucleic acid molecule encoding a suitable cytokine, a promoter molecule of choice, including, without limitation, enhancers, and leader sequences; a suitable 3′ regulatory region to allow transcription in the host, and any additional desired components, such as reporter or marker genes, are cloned into the vector of choice using standard cloning procedures in the art, such as described in Sambrook et al., Molecular Cloning: A Laboratory Manual, Cold Spring Laboratory, Cold Spring Harbor, N.Y. (1989); Ausubel et al., “Short Protocols in Molecular Biology,” New York:Wiley (1999), and U.S. Pat. No. 4,237,224 to Cohen and Boyer, which are hereby incorporated by reference in their entirety.

Once the isolated nucleic acid molecule has been cloned into an expression vector, it is ready to be incorporated into a host. Recombinant molecules can be introduced into cells, without limitation, via transfection (if the host is a eukaryote), transduction, conjugation, mobilization, or electroporation, lipofection, protoplast fusion, mobilization, or particle bombardment, using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, and mammalian cells, including, without limitation, stem cells and CD11c⁺ dendritic cells.

Depending upon whether then cell to be transformed, e.g., the CD11c⁺ dendritic cell or CD4+ T cell, is ex vivo or in vivo, different modes of introducing the expression vector can be carried out.

Transient expression in protoplasts allows quantitative studies of gene expression since the population of cells is very high (on the order of 106). To deliver DNA inside protoplasts, several methodologies have been proposed, but the most common are electroporation (Neumann et al., “Gene Transfer into Mouse Lyoma Cells by Electroporation in High Electric Fields,” EMBO J 1:841-45 (1982); Wong et al., “Electric Field Mediated Gene Transfer,” Biochem Biophys Res Commun 30:107(2):584-7 (1982); Potter et al., “Enhancer-Dependent Expression of Human Kappa Immunoglobulin Genes Introduced into Mouse pre-B Lymphocytes by Electroporation,” Proc. Natl. Acad. Sci. USA 81:7161-65 (1984, which are hereby incorporated by reference in their entirety) and polyethylene glycol (PEG) mediated DNA uptake, Sambrook et al., Molecular Cloning: A Laboratory Manual, Chap. 16, Second Edition, Cold Springs Laboratory, Cold Springs Harbor, N.Y. (1989), which is hereby incorporated by reference in its entirety). During electroporation, the DNA is introduced into the cell by means of a reversible change in the permeability of the cell membrane due to exposure to an electric field. PEG transformation introduces the DNA by changing the elasticity of the membranes. Unlike electroporation, PEG transformation does not require any special equipment and transformation efficiencies can be equally high. Another appropriate method of introducing the nucleic acid construct of the present invention into a host is fusion of protoplasts with other entities, either minicells, cells, lysosomes, or other fusible lipid-surfaced bodies that contain the chimeric gene (Fraley, et al., Proc Natl Acad Sci USA 79:1859-63 (1982), which is hereby incorporated by reference in its entirety).

Stable transformants are preferable for the methods of the present invention, which can be achieved by using variations of the methods above as describe in Sambrook et al., Molecular Cloning: A Laboratory Manual, Chap. 16, Second Edition, Cold Springs Laboratory, Cold Springs Harbor, N.Y. (1989), Ausubel et al., “Short Protocols in Molecular Biology,” New York:Wiley (1999), and U.S. Pat. No. 4,237,224 to Cohen and Boyer, which are hereby incorporated by reference in their entirety. For in vivo transformation, naked DNA, infective delivery vehicles, and non-infective delivery vehicles are preferred. Use of naked DNA and non-infective delivery vehicles can be particularly useful when only transient expression is desired, whereas infective delivery vehicles are preferred for stable transformation.

The therapeutic nucleic acid molecules can be administered via a liposomal delivery mechanism. Basically, this involves providing a liposome which includes the siRNA or expression vector to be delivered, and then contacting the target cell or tissues with the liposome under conditions effective for delivery of its payload into the cell.

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), each of which is hereby incorporated by reference in its entirety). When liposomes are endocytosed by a target cell, for example, they can be routed to acidic endosomes which will destabilize the liposome and result in drug release.

The liposome delivery system can also be made to accumulate at a target organ, tissue, or cell via active targeting (e.g., by incorporating an antibody or hormone on the surface of the liposomal vehicle). This can be achieved according to known methods.

Different types of liposomes can be prepared according to Bangham et al., J. Mol. Biol. 13:238-252 (1965); U.S. Pat. No. 5,653,996 to Hsu et al.; U.S. Pat. No. 5,643,599 to Lee et al.; U.S. Pat. No. 5,885,613 to Holland et al.; U.S. Pat. No. 5,631,237 to Dzau et al.; and U.S. Pat. No. 5,059,421 to Loughrey et al., each of which is hereby incorporated by reference.

These liposomes can be produced such that they contain, in addition to the therapeutic nucleic acid molecule, other therapeutic agents, such as anti-inflammatory agents, which would then be released at the target site (e.g., Wolff et al., Biochem. et Biophys. Acta 802:259 (1984), which is hereby incorporated by reference in its entirety).

As an alternative to non-infective delivery of the inhibitory RNA as described above, naked DNA or infective transformation vectors can be used for delivery, whereby the naked DNA or infective transformation vector contains a recombinant gene that encodes the therapeutic nucleic acid or protein or polypeptide capable of modulating osteoclastogenesis of CD11c⁺DC. The therapeutic nucleic acid molecule is then expressed in the transformed cell.

The recombinant gene includes, operatively coupled to one another, an upstream promoter operable in mammalian cells and optionally other suitable regulatory elements (i.e., enhancer or inducer elements), a coding sequence that encodes the therapeutic nucleic acid (described above), and a downstream transcription termination region. Any suitable constitutive promoter or inducible promoter can be used to regulate transcription of the recombinant gene, and one of skill in the art can readily select and utilize such promoters, whether now known or hereafter developed. Tissue specific promoters can also be used. One suitable approach for inducible/repressible promoters involves use of a TetO response element. Other inducible elements can also be used. Known recombinant techniques can be utilized to prepare the recombinant gene, transfer it into the expression vector (if used), and administer the vector or naked DNA to a patient. Exemplary procedures are described in Sambrook et al., Molecular Cloning: A Laboratory Manual, Second Edition, Cold Spring Harbor Press, NY (1989), which is hereby incorporated by reference in its entirety. One of skill in the art can readily modify these procedures, as desired, using known variations of the procedures described therein.

Any suitable viral or infective transformation vector can be used. Exemplary viral vectors include, without limitation, adenovirus, adeno-associated virus, and retroviral vectors (including lentiviral vectors).

Adenovirus gene delivery vehicles can be readily prepared and utilized given the disclosure provided in Berkner, Biotechniques 6:616-627 (1988) and Rosenfeld et al., Science 252:431-434 (1991), WO 93/07283, WO 93/06223, and WO 93/07282, each of which is hereby incorporated by reference in its entirety. Additional types of adenovirus vectors are described in U.S. Pat. No. 6,057,155 to Wickham et al.; U.S. Pat. No. 6,033,908 to Bout et al.; U.S. Pat. No. 6,001,557 to Wilson et al.; U.S. Pat. No. 5,994,132 to Chamberlain et al.; U.S. Pat. No. 5,981,225 to Kochanek et al.; U.S. Pat. No. 5,885,808 to Spooner et al.; and U.S. Pat. No. 5,871,727 to Curiel, each of which is hereby incorporated by reference in its entirety.

Adeno-associated viral gene delivery vehicles can be constructed and used to deliver into cells a recombinant gene encoding a desired nucleic acid. The use of adeno-associated viral gene delivery vehicles in vitro is described in Chatterjee et al., Science 258:1485-1488 (1992); Walsh et al., Proc. Nat'l Acad. Sci. USA 89:7257-7261 (1992); Walsh et al., J. Clin. Invest. 94:1440-1448 (1994); Flotte et al., J. Biol. Chem. 268:3781-3790 (1993); Ponnazhagan et al., J. Exp. Med. 179:733-738 (1994); Miller et al., Proc. Nat'l Acad. Sci. USA 91:10183-10187 (1994); Einerhand et al., Gene Ther. 2:336-343 (1995); Luo et al., Exp. Hematol. 23:1261-1267 (1995); and Zhou et al., Gene Ther. 3:223-229 (1996), each of which is hereby incorporated by reference in its entirety. In vivo use of these vehicles is described in Flotte et al., Proc. Nat'l Acad. Sci. USA 90:10613-10617 (1993); and Kaplitt et al., Nature Genet. 8:148-153 (1994), each of which is hereby incorporated by reference in its entirety.

Retroviral vectors which have been modified to form infective transformation systems can also be used to deliver a recombinant gene encoding a desired nucleic acid product into a target cell. One such type of retroviral vector is disclosed in U.S. Pat. No. 5,849,586 to Kriegler et al., which is hereby incorporated by reference in its entirety. Lentivirus vectors can also be utilized, including those described in U.S. Pat. No. 6,790,657 to Arya, and U.S. Patent Application Nos. 20040170962 to Kafri et al. and 20040147026 to Arya, each of which is hereby incorporated by reference in its entirety.

The various delivery systems can be administered to a patient via direct injection into the affected tissues (e.g., bone compartment, bone marrow, etc.) or via systemic administration. System modes of administration include, without limitation, orally, by inhalation, by intranasal instillation, topically, transdermally, parenterally, subcutaneously, intravenous injection, intra-arterial injection (such as via the pulmonary artery), intramuscular injection, intrapleural instillation, intraperitoneally injection, intraventricularly, intralesionally, by application to mucous membranes, or implantation of a sustained release vehicle.

Another aspect of the present invention is the treatment of an inflammatory, metabolic, or genetic bone diseases or disorders, as well as bone tumors (e.g., osteosarcoma). Basically, the methods of upregulating or downregulating osteoclastogenesis via CD11c⁺DCs can be practiced for the purpose of promoting or inhibiting osteoclastogenesis to treat diseases or disorders associated with excessive bone deposition or excessive bone resorption, respectively. Examples of diseases or disorders characterized by excessive deposition include, without limitation, osteopetrosis and osteoarthritis. Examples of diseases or disorders characterized by excessive resorption include, without limitation, osteoporosis, osteomyelitis, rheumatoid arthritis, periodontal disease, and Pagets Disease.

By treating, it is intended to the therapeutic uses of the present invention can provide temporary or, with continued use, permanent relief of symptoms associated with the bone disease or disorder. In other words, it is expected that the therapies associated with the present invention will slow or prevent worsening of symptoms or reduce the severity of symptoms (i.e., reversal of undesired bone loss or cessation of undesired bone deposition).

For the various therapeutic uses, standard aseptic cell recovery methods well-known in the art are preferably used to harvest CD11c⁺ and/or CD4⁺ T cells. The cells may be removed from any number of sources known to contain myeloid stem cell precursors including, without limitation, the subject's bone marrow, peripheral blood, spleen, liver, and lymph nodes.

To promote osteoclastogenesis (and bone resorption), one or both types of cells can be treated in accordance with the present invention (described supra) to promote osteoclastogenesis of the CD11c⁺ dendritic cells. This can also be achieved by genetic manipulation of either the CD11c⁺ dendritic cells or the CD4⁺ T cells, whereby upon return to the patient's body, osteoclastogenesis of the CD11c⁺ dendritic cells is promoted. The genetic manipulation can include one or more suitable nucleic acid molecules encoding a bone modulating cytokine, chemokine, growth hormone, antibody, or protein or polypeptide capable of promoting osteoclastogenesis in CD11c⁺ dendritic cells.

To inhibit osteoclastogenesis (slowing resorption and possibly promoting deposition), either the CD11c⁺ dendritic cells or the CD4⁺ T cells can be genetically manipulated, whereby upon return to the patient's body, osteoclastogenesis of the CD11c⁺ dendritic cells is inhibited. The genetic manipulation can involve expression of inhibitory nucleic acid molecules that reduces or inhibits expression of a bone modulating cytokine, chemokine, growth hormone, antibody, or protein or polypeptide that normally promotes osteoclastogenesis.

Alternatively, the isolated CD11c⁺ dendritic cells may be directly stimulated by with a bone modulating factor. Suitable bone modulating factors of the present invention include, without limitation, cytokines, chemokines, growth hormones, antibodies (e.g., anti-RANKL antibody), or proteins or polypeptides, including those described herein above. This can be accomplished ex vivo (during culturing of the CD11c⁺ dendritic cells) or in vivo.

In yet another aspect of the present invention, the disease or disorder may be one in which a pathogen is implicated in the disease. In this aspect, the disease is treated by removing CD11c⁺ dendritic cells from a subject as described above, treating the CD11c⁺ dendritic cells with a pathogen-specific cell surface ligand, and returning the treated CD11c⁺ dendritic cells to the subject, thereby treating the disease or disorder. The pathogen-specific cell surface ligand is specific for a pathogen implicated in the inflammatory bone disease or metabolic bone disorder. Such pathogen-specific ligand useful in this aspect of the present invention include, without limitation, ligands specific for A. actinomycetemcomitans and P. gingivalis.

The methods of modifying CD11c⁺ dendritic cell differentiation to form osteoclasts can be used to screen for compounds that affect myeloid osteoclastogenesis. This involves the ex vivo culturing procedures described above; exposing the dendritic cells or osteoclast precursor cells to a compound, and determining the ability of the compound of affect myeloid osteoclastogenesis. Stimulation or inhibition of osteogenesis by a test compound can determined by comparing the test culture with a culture of CD11c⁺ dendritic cells grown under control conditions. Testing for osteogenesis can be carried by various methods known in the art, including osteoclast cell count (e.g., using OC-specific staining); TRAP staining, resorption assays, or a combination thereof.

Another aspect of the present invention is a method of identifying genes related to the production of functional osteoclasts from CD11c⁺ dendritic cells. This method involves the ex vivo culturing procedures described above, where one or more of the culture conditions are modified. Following a suitable time in culture under the altered conditions, osteoclast production and function of the cells in the culture are determined, and the cells are screened for one or more genes associated with the change in osteoclast production or function in the culture (as compared with a culture in which culture conditions were not altered). Osteoclast production may be determined by counting the number of TRAP positive cells, osteoclast function may be determined by bone resorption assay. The screening for the gene or genes associated with change in osteoclast formation or function is carried out using any gene methods known in the art, including, without limitation, differential gene display, RNA arbitrarily primed (RAP)-PCR, or gene microarray analysis.

Some aspects of the present invention involve using an isolated antibody, or binding portion thereof, against a bone modulating cytokine, chemokine, growth hormone, or a nucleic acid molecule encoding such an antibody or binding portion thereof. Thus, this aspect of the present invention involves producing antibodies against (i.e., that recognize) a bone modulating cytokine, chemokine, growth hormone, antibody, or protein or polypeptide useful in this and other aspects of the present invention. The antibodies can be monoclonal or polyclonal.

Monoclonal antibody production may be effected by techniques which are well-known in the art. Basically, the process involves first obtaining immune cells (lymphocytes) from the spleen of a mammal (e.g., mouse) which has been previously immunized with the antigen of interest either in vivo or in vitro. The antibody-secreting lymphocytes are then fused with (mouse) myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, “Continuous Culture of Fused Cells Secreting Antibody of Predefined Specificity,” Nature, 256:495-7 (1975), which is hereby incorporated by reference in its entirety.

Mammalian lymphocytes are immunized by in vivo immunization of the animal (e.g., a mouse) with a selected antigen. Such immunizations are repeated as necessary at intervals of up to several weeks to obtain a sufficient titer of antibodies. Following the last antigen boost, the animals are sacrificed and spleen cells removed.

Fusion with mammalian myeloma cells or other fusion partners capable of replicating indefinitely in cell culture is effected by standard and well-known techniques, for example, by using polyethylene glycol (“PEG”) or other fusing agents (Milstein et al., “Derivation of Specific Antibody-Producing Tissue Culture and Tumor Lines by Cell Fusion,” Eur. J. Immunol., 6:511-19 (1976), which is hereby incorporated by reference in its entirety). This immortal cell line, which may be derived from cells of any mammalian species, including, but not limited to, mouse, rat, and human, is selected to be deficient in enzymes necessary for the utilization of certain nutrients, to be capable of rapid growth, and to have good fusion capability. Many such cell lines are known to those skilled in the art, and others are regularly described.

Procedures for raising polyclonal antibodies are also well known. Typically, such antibodies can be raised by administering the selected antigen subcutaneously to New Zealand white rabbits which have first been bled to obtain pre-immune serum. The antigens can be injected at a total volume of 100 μl per site at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthanized with pentobarbital 150 mg/Kg IV. This and other procedures for raising polyclonal antibodies are disclosed in E. Harlow, et. al., Editors, Antibodies: a Laboratory Manual (1988), which is hereby incorporated by reference in its entirety.

An example of an antibody that can inhibit osteoclastogenesis is anti-RANKL.

### EXAMPLES

The following examples are provided to illustrate embodiments of the present invention but they are by no means intended to limit its scope.

### Materials and Methods

Mice: 4-6 wk old female Balb/c, B6C3fe Csf-1^{-/-}op/op and wild type (WT) control mice were purchased from the JAX Mice (Bar Harbour, Me.) and housed under specific pathogen-free conditions in the animal facilities of the University of Rochester, Rochester, N.Y. and the University of Western Ontario, Ontario, Canada. All animal protocols were conducted under each institution's guidelines and approved by the local Ethics and Animal Experimentation Committees.

Cell cultures and reagents: All primary cell cultures were performed in complete RPMI-1640 media supplemented with 10% heat inactivated fetal bovine serum (GIBCO, Toronto), 50 μM β2-mercaptoethanol, 100 μg/ml streptomycin, and 100 U/ml penicillin. Cells were incubated at 37° C. in a humidified 5% CO₂ incubator (Teng et al, “Functional Human T-Cell Immunity and Osteoprotegerin-Ligand (OPG-L) Control Alveolar Bone Destruction in Periodontal Infection,” J. Clin. Invest. 106:R59-R67 (2000), which is hereby incorporated by reference in its entirety). The following reagents were purchased from commercial sources: rmGM-CSF (Cedarlane, ON), anti-mFcRIII/II (CD16/32), PE-conjugated, anti-mCD4, FITC-conjugated anti-mCD11c, PerCPCy5.5-conjugated anti-mCD4, rat anti-mI-A^{d}, biotin anti-mCD31 (ER-MP12), biotin anti-mLy-6C (ER-MP20), rat anti-mTCR αβ chain, rat anti-mCD3Abs, rmIL-4, Concanavalin A (ConA) were all from BD Pharmingen, (Toronto, ON), FITC-conjugated goat anti-hFc-γ (Jackson Labs, ME), magnetic beads-conjugated anti-mCD11c (Miltenyi Biotec, CA), anti-mM-CSF Ab (R&D Systems, MN), anti-mTRAP Ab (Zymed, MA), rabbit anti-mRANK, rabbit anti-mGM-CSFR, goat anti-mCT-R and bovine anti-goat Texas red-conjugated IgG, goat anti-m cathepsin k Abs (Santa Cruz Biotech, CA), rabbit & goat isotype matched IgGs, mrM-CSF, FITC-conjugated goat anti-human Fc specific IgG, bovine serum albumin fraction V (BSA) and LPS from E. coli 0128:B12 (Sigma), biotinylated goat anti-rabbit IgG, Streptavidin-AMCA (7 amino-methylcoumarin) and Streptavidin-PE (Vector Lab, CA), anti-mCD11b-Cy5, anti-m-integrin β₃ (CD61)-FITC and biotinylated anti-m-integrin αᵥ (CD51) [clone RMV-7], biotinylated goat anti-Armenian hamster, biotin anti-mF4/80 and biotinylated anti-mCD25 Abs (eBioscience, CA), Alexa Fluor 488 phalloidin (Molecular Probes, OR). rmRANKL and OPG-Fc fusion proteins were prepared as previously described (Kong et al., “OPGL is a Key Regulator of Osteoclastogenesis, Lymphocyte Development and Lymph-Node Organogenesis,” Nature 397:315-323 (1999); Teng et al, “Functional Human T-Cell Immunity and Osteoprotegerin-Ligand (OPG-L) Control Alveolar Bone Destruction in Periodontal Infection,” J. Clin. Invest. 106:R59-R67 (2000), each of which is hereby incorporate by reference in its entirety). Quick cell proliferation assay kit (WST-1; BioVision, CA).

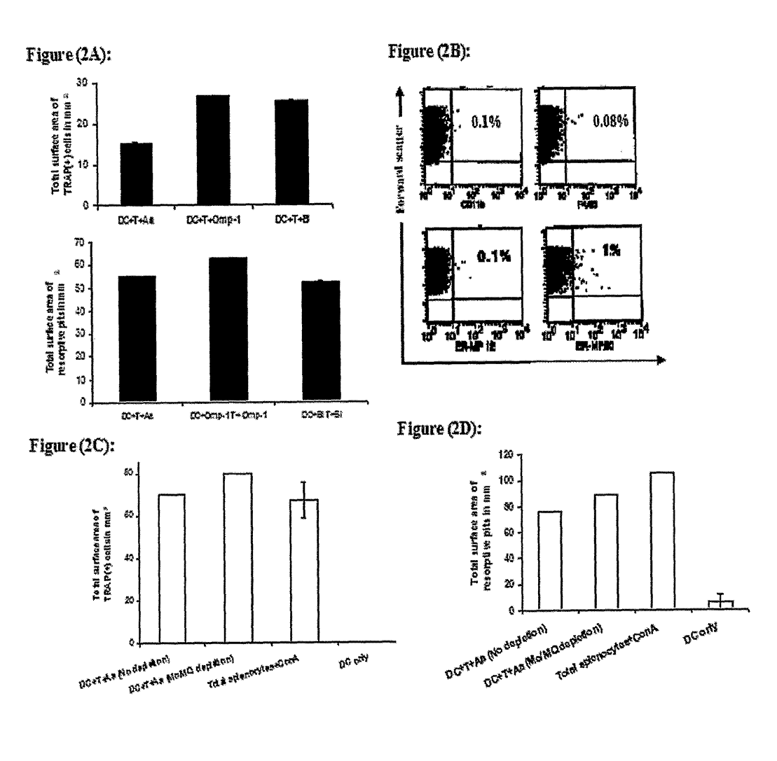

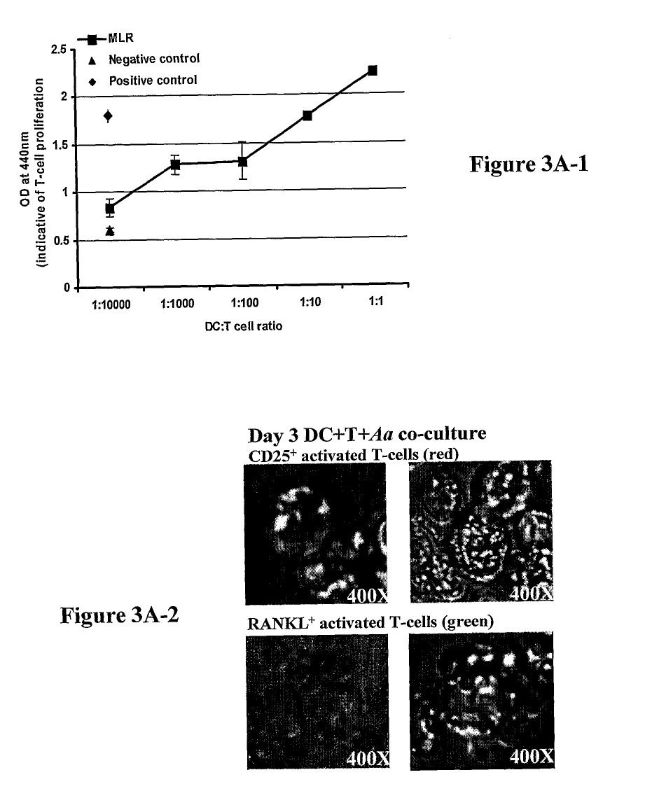

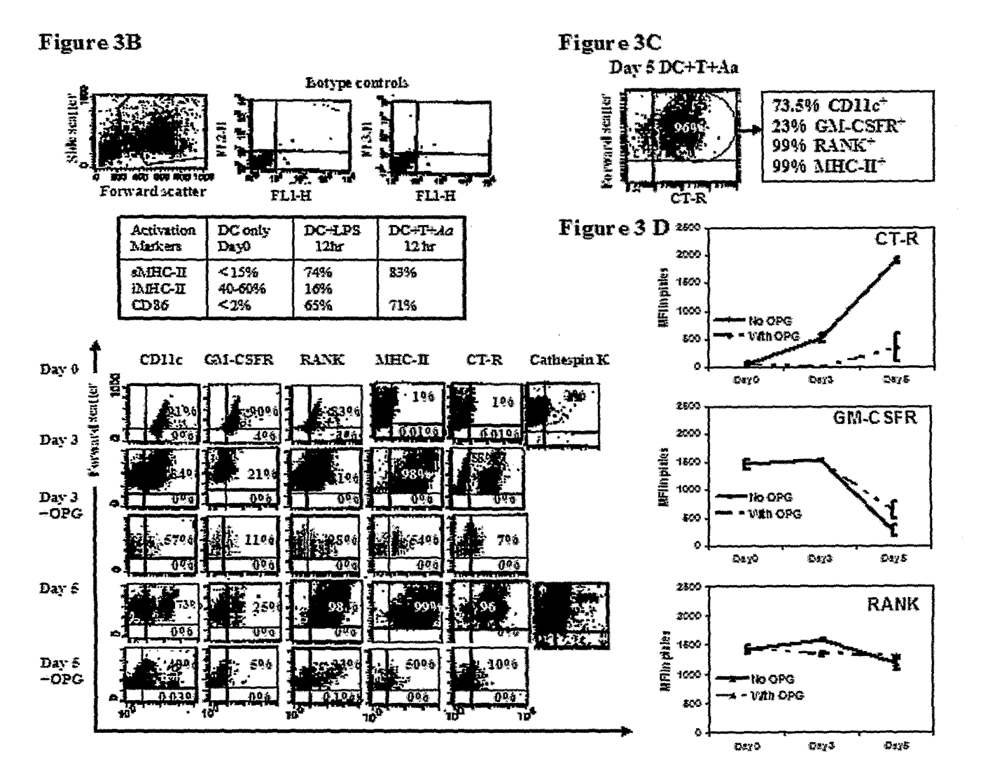

Generation of BM-derived CD11c⁺DC and CD11c⁺DC-CD4⁺T-cells co-cultures: CD11c⁺DC were generated from BM of Balb/c, B6C3fe Csf-1^{op/op} and WT control mice using a previously described protocol (Inaba et al., “Generation of Large Numbers of Dendritic Cells from Mouse Bone Marrow Cultures Supplemented with Granulocyte/Macrophage Colony-Stimulating Factor,” J. Exp. Med. 176:1693-1702 (1992), which is hereby incorporated by reference in its entirety) with modification. Briefly, total BM cells freshly isolated from long bones, were cultured with 20 ng/ml rmGM-CSF and 10 ng/ml rmIL-4. To avoid contamination by typical CD11b⁺ myeloid subsets (i.e., OC precursors) and to enrich for a pure DC population, MACS sorting was applied (Miltenyi Biotec, CA). On day 7, CD11c⁺DC were purified with magnetic bead conjugated-mCD11c mAb using MACS sorter. The resulting population is highly pure as CD11c⁺DC purity is >99% (see FACS data in FIG. 3B), constituting ≈10-15% of the total “numbers” of BM-derived DC after 1 wk culture with GM-CSF & IL-4. In particular, this subset is CD11b⁻ (see FIG. 2B), different from the rather heterogeneous BM-derived “un-sorted” DC cultures consisting of CD11c⁺DC that is mostly CD11b⁺ (Schuler, “Chapter 27: A Guide to the Isolation and Propagation of Dendritic Cells,” In Dendritic Cells: Biology and Clinical Applications, Lotze et al. (eds.), San Diego, Calif., Academic, pp. 515-533 (1999); Inaba et al., “Generation of Large Numbers of Dendritic Cells from Mouse Bone Marrow Cultures Supplemented with Granulocyte/Macrophage Colony-Stimulating Factor,” J. Exp. Med. 176:1693-1702 (1992), each of which is hereby incorporated by reference in its entirety). In some experiments, CD11c⁺DC were generated after depleting Mo and MQ cells via Ab-dependent complement-mediated lysis. Briefly, BM cells were first incubated with Abs against Mo markers Ly6C (ER-MP20) and CD31 (ER-MP12); and MQ markers F4/80 and CD11b; of 3-5 μg/ml each, for 30 min followed by the addition of rabbit complement and incubation at 37° C. for 30 min (Teng et al, “Functional Human T-Cell Immunity and Osteoprotegerin-Ligand (OPG-L) Control Alveolar Bone Destruction in Periodontal Infection,” J. Clin. Invest. 106:R59-R67 (2000); Teng et al., “Evidence for Th2 Cell-Mediated Suppression of Antibody Response in Transgenic Beef Insulin-Tolerant Mice,” Eur. J. Immunol. 25:2522-2527 (1995); Leenen et al., “Murine Macrophage Precursor Characterization II. Monoclonal Antibodies Against Macrophage Precursor Antigens,” Eur. J. Immunol. 20:27-34 (1990), each of which is hereby incorporate by reference in its entirety). After lysis, remaining Mo/MQ depleted BM cells were re-suspended in conditioned media with mGM-CSF and mIL-4 for 7 days and subsequently CD11c⁺DC were MACS purified.

To generate CD4⁺T-cells, total splenocytes were prepared from naïve syngeneic Balb/c mice after lysis of RBC. Splenocytes were passed through a nylon-wool column to enrich T-cells, after which CD4⁺ T-cells were further purified via direct panning on an anti-mCD4-GK1.5 mAb-coated Petri dish, the yielded CD4⁺T-cells were 95-97% pure (Teng, et al, “Functional Human T-Cell Immunity and Osteoprotegerin-Ligand (OPG-L) Control Alveolar Bone Destruction in Periodontal Infection” J. Clin. Invest. 106:R59-R67 (2000), which is hereby incorporated by reference in its entirety). Purified 0.5×10⁶ CD11c⁺DC were co-cultured with 0.5×10⁶ CD4⁺T-cells on dentine slices, synthetic bone discs (BD BIOCOAT™, OSTEOLOGIC™), or HA-coated wells (OCT, USA) in triplicate with or without the following reagents: Aa sonicate antigens (10 μg/ml), ConA (5 μg/ml; 17), Aa-OPM-1 antigen or bovine insulin-BI (20 μg/ml), rmRANKL (30 ng/ml), OPG (10-30 μg/ml), rabbit anti-mRANK, rat anti-mMHC-II, rat anti-mTCR α chain, rat anti-mCD3 [symbol] goat anti-mM-CSF (10 μg/ml) Abs, in HA-coated 48-well or dentine-slices flat-bottom plates. Due to scant CD11c⁺DC obtained from Csf-1^{op/op} mice BM cultures, only 10⁵ Csf-1^{-/-}op/op CD11c⁺DC were co-cultured with 3×10⁵ CD4⁺T-cells or rmRANKL in HA plates with or without rmM-CSF (25 ng/ml). DC co-cultures were incubated up to 5-10 days during which DC were checked for TRAP and resorptive pits (lacunae) activities, as well as surface phenotype by flow cytometry and immuno-fluorescent microscopy. DC viability was assessed by trypan-blue exclusion during the co-culture period. 0.5×10⁶ splenocytes stimulated by ConA for 2-3 days (Horwood et al., “Activated T-cells Support Osteoclast Formation in vitro,” Biochem. Biophys. Res Communs. 256:144-150 (1999), which is hereby incorporated by reference in its entirety) were used as positive controls for TRAP and resorptive pit assays.

Quantitation of TRAP⁽⁺⁾ cells and resorptive pits in HA co-cultures: Co-cultured CD11c⁺DC were fixed by 2% formaldehyde+0.2% glutaraldehyde in PBS, pH 7.3, for 45 min. Cells were then incubated with TRAP staining solution (0.2 sodium acetate buffer, 40 mM sodium tartrate, 1.2 mM napthol AS-MX phosphate, and 1.3 mM fast red violet LB salt). To quantify TRAP signals, images were digitally captured under 400× magnification using Leica inverted microscope IRBE-DM via a motorized staging facility equipped with a high resolution Hamamatsu-Orca digital camera (Mahamed et al., “G(−) Anaerobes-Reactive CD4⁺ T-Cells Trigger RANKL-Mediated Enhanced Alveolar Bone Loss in Diabetic NOD Mice,” Diabetes 54:1477-1486 (2005), which is hereby incorporated by reference in its entirety). Both total surface areas and numbers of (purple-red) TRAP(+) DDOC were quantified as previously described (Mahamed et al., “G(−) Anaerobes-Reactive CD4⁺ T-Cells Trigger RANKL-Mediated Enhanced Alveolar Bone Loss in Diabetic NOD Mice,” Diabetes 54:1477-1486 (2005), which is hereby incorporated by reference in its entirety). Briefly, 12-17 random fields per well were picked for automated scanning analysis. The mean of total surface area of TRAP(+) DC was determined after subtracting averaged background signals from the negative control. To quantify the total surface area of resorptive pits per well, cells were stripped by 1N NaOH for 16 hrs, after which the images of eroded HA surfaces were captured as described above.

Mixed lymphocyte culture: Naïve 3×10⁵ CD4⁺T-cells purified from C57BL/6 splenocytes were co-cultured, in triplicate, with different densities (3×10⁵, 3×10⁴, 3×10³, 3×10², 3×10¹) of freshly purified CD11c⁺DC derived from BM of Balb/c mice as described above in flat-bottom 96 well plate in a total volume of 100 μl. Plate bound anti-CD3 Ab (10 μg/ml) activated CD4⁺T-cells were used as positive control for T-cell proliferation, while untreated CD4⁺T-cells were used as a negative control. At day 5, 50 μl of WST-1 reagent was added into each well according to the manufacturer's instructions for colorimetric measurement of cellular proliferation (Pan et al., “Effect of Copper Deficiency on Oxidative DNA Damage in Jurkat T-Lymphocytes,” Free Radic. Biol. Med. 28(5):824-830 (2000), which is hereby incorporated by reference in its entirety). This assay is based on the cleavage of tetrazolium salt WST-1 to formazan by mitochondrial dehydrogenase. OD value was determined 4 hrs later at 440 nm using Dynatech MR700 Micro-plate reader (Dynatech, VA).

FACS analysis and Scanning Immunofluorescent Digital Images Analysis (SIDIA): Cells collected from co-cultures at various time points were incubated with 5 μg/ml anti-CD16/32 to block background binding, then immuno-stained with isotype control IgG or Ab-conjugated to FITC, PE, Cy5, or biotin as follows: cells were stained with FITC-anti-mCD11c, anti-mGM-CSFR, anti-mRANK Ab followed by incubation with 2° (biotinylated anti-rabbit IgG) or 3°-streptavidin-PE or cy5; anti-mCT-R IgG followed by 2° FITC-conjugated anti-goat IgG; anti-m I-A^{d} followed by 2°-biotin goat anti-mIgG and 3°-streptavidin-Cy5. For intracellular staining of cathepsin-k, cells were fixed for 30 min with fresh 4% formaldehyde, and then permealized for 10 min with 0.2% Triton-X100 followed by blocking non-specific binding by 1% BSA and incubation with anti-m-cathepsin-k IgG and 2°-FITC-conjugated anti-goat IgG on ice. Stained cells were scanned in a FACS-CALIBUR™ flow cytometer (BD Biosciences). Results were analyzed via CELLQUEST™ and dead cells excluded by PI staining. CD4⁺ T-cell activation was assessed by in situ staining of CD25 with 1° biotinylated anti-mCD25, 2° streptavidin-Cy5 and RANKL with 1° OPG-Fc, 2° FITC conjugated anti-human Fc IgG. While intracellular staining of F-actin was performed using Alexa Fluor 488 phalloidin according to the manufacturer's protocol. For quantitation of immunofluorescent (IF) signals at the single cell level, 0.25×10⁶ CD11c⁺DC were labelled by carboxyfluorescein diacetate succinimidyl ester (CFSE) before co-cultures; then at different time points were stained with: i) anti-mCT-R and 2° Texas red-conjugated anti-goat IgG, and anti-GM-CSFR using biotinylated IgG, Streptavidin-AMCA (7 amino-methylcoumarin) as 2°- and 3°-Abs, ii) CT-R (Texas Red) and anti-RANK using biotinylated IgG, Streptavidin-AMCA as 2° and 3° Abs. Stained DC were then placed in 96-well flat bottom plates for SIDIA. IF images were captured for a series of 12-17 random fields per well after which the mean FI (MFI) at single cell level was determined as previously described excluding all CFSE-negative cells (<50 pixels; 20). The sum of FI was calculated by adding the total FI values per well after subtracting the background signals. The results are shown as the mean±S.E.M. of FI by at least 0.1×10⁶ countable cells from 3 independent experiments.

Adoptive transfer of CD11c⁺DC onto mouse calvaria and bone resorption: Purified CD11c⁺DC were stained with CFSE prior to co-cultures with CD4⁺ T-cells and Aa. On day 2.5, DC were collected and stained with anti-mCD11c mAb plus biotin-conjugated anti-hamster IgG and streptavidin-conjugated Cy5. Then, 2×10⁶ CD11c⁺DC (100 μl PBS) was injected onto the calvarias of 5-6 wk old female NOD/SCID mice (n=3). NOD/SCID mice receiving PBS only, 2×10⁶ CD11c⁺DC with or without Aa, or CD4⁺ T-cells with or without Aa (n=3) served as negative controls. All mice were euthanized 4 days post injection. The injected cells were detected in vivo on mouse skulls when scanned using two sets of fluorescent filters to detect CFSE- (465 nm-excitation/545 nm-emission) and Cy5-(620 nm/670 nm) via a Kodak image-station 2000 MM. Skulls were fixed in 37% formalin and decalcified in Cal-EX to prepare 4-6 μm-thick tissue sections for histology, and immunohistochemistry for TRAP detection (Teng et al, “Functional Human T-Cell Immunity and Osteoprotegerin-Ligand (OPG-L) Control Alveolar Bone Destruction in Periodontal Infection,” J. Clin. Invest. 106:R59-R67 (2000); Teng et al., “Periodontal Immune Responses of Human Lymphocytes in Actinobacillus actinomycetemcomitans-Inoculated NOD/SCID Mice Engrafted with Peripheral Blood Leukocytes of Periodontitis Patients,” J. Periodontal Res. 1:54-61 (1999), each of which is hereby incorporate by reference in its entirety). Fluorescent microscopy was utilized to visualize CFSE-labelled DC in histological sections.

Statistical analysis: Statistical analysis was performed using the two-sided Student t-test. Differences between groups were considered statistically significant with >95% confidence when p value was <0.05.

### Example 1

### CD11c⁺DC Develop TRAP and Bone Resorptive Activities During Co-Culture with CD4⁺T-Cells and Foreign Antigens in a RANKL Dependent Manner

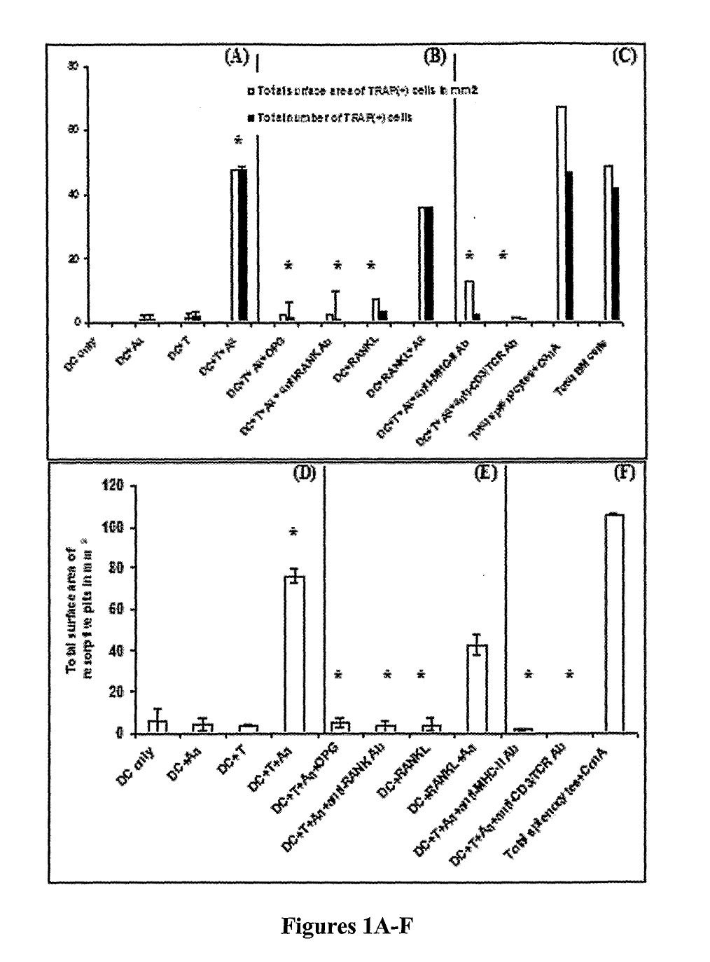

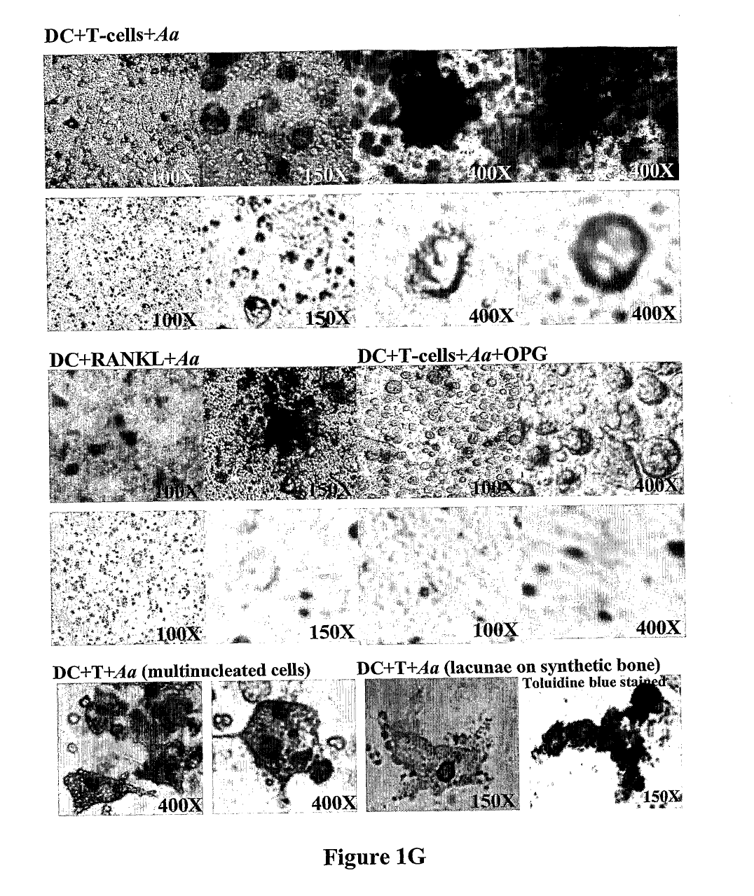

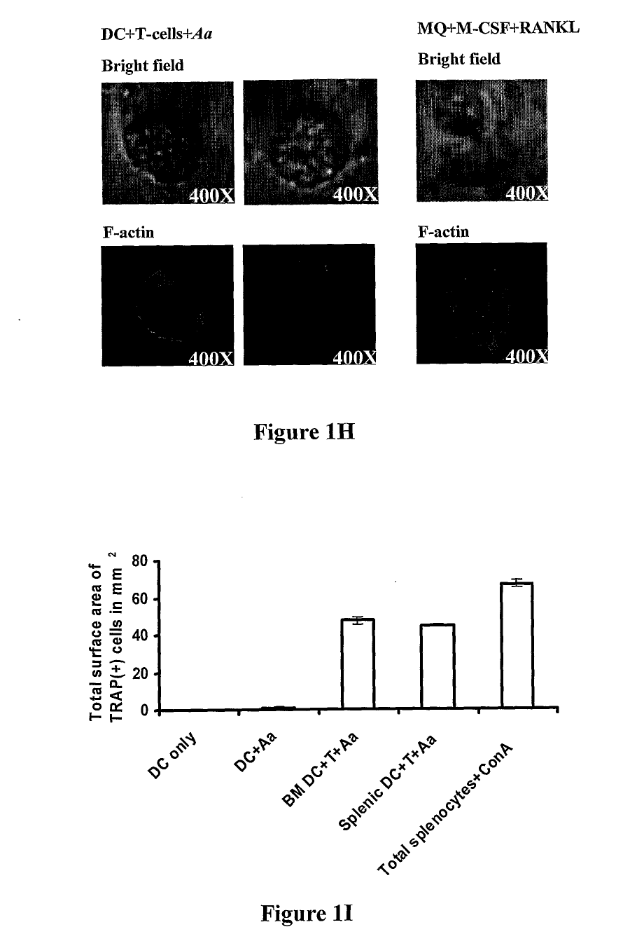

To explore the osteoclastogenic potential of DC, Balb/c BM-derived MACS purified CD11c⁺DC were incubated with naïve syngeneic CD4⁺ T-cells (in 1:1 up to 1:10 ratio) and sonicated antigens of Aa, a key human periodontal pathogen (Teng et al, “Functional Human T-Cell Immunity and Osteoprotegerin-Ligand (OPG-L) Control Alveolar Bone Destruction in Periodontal Infection,” J. Clin. Invest. 106:R59-R67 (2000); Mahamed et al., “G(−) Anaerobes-Reactive CD4⁺ T-Cells Trigger RANKL-Mediated Enhanced Alveolar Bone Loss in Diabetic NOD Mice,” Diabetes 54:1477-1486 (2005), each of which is hereby incorporate by reference in its entirety), on dentine slices, synthetic bone discs or hydroxyapatite (HA)-coated 48-well plates for 2-10 days. Total splenocytes plus ConA co-cultures were used as positive control for TRAP and resorptive activity (Horwood et al., “Activated T-cells Support Osteoclast Formation in vitro,” Biochem. Biophys. Res Communs. 256:144-150 (1999), which is hereby incorporated by reference in its entirety), and freshly prepared TRAP stained total BM cells were used to validate the positive control results (FIG. 1C). Interestingly, CD11c⁺DC were able to fuse and become TRAP(+) multinucleated giant cells, some of which manifested dendrites and maintained interaction with T-cells (FIG. 1G). Since T-cells do not express TRAP (confirmed by TRAP staining of CD4⁺ T-cells co-cultured with and without Aa or ConA), all TRAP(+) cells must be DC-derived. TRAP activity on day 5 was assessed by the total surface area and the total number of TRAP(+) cells (FIG. 1A). More than 95% of TRAP(+) cells included in the quantitative analysis were multinucleated (≧3 nuclei). Titration of cells to as low as 5×10⁴ CD11c⁺DC and 5×10⁴ CD4⁺ T-cells also yielded significant TRAP and resorptive pit results compared to negative controls.

TRAP expression peaked on day 5 and required stimulation by Aa, as DC cultured alone, DC plus Aa, DC plus T-cells without Aa, did not develop significant TRAP activity (p≦0.03; FIG. 1A). Surprisingly, as early as day 2, total surface area and numbers of TRAP(+) cells were already significantly higher than negative controls (i.e. DC alone, DC+Aa, & DC+T), which waned by day 8-9.

Subsequently, cells were stripped and images of bone surfaces were captured and quantified to determine whether TRAP(+) multinucleated cells formed resorptive pits. The results showed that total surface area of resorptive pits in DC, CD4⁺T-cells and Aa co-cultures were significantly higher than negative controls (FIG. 1D: p≦0.03), indicating that these cells can indeed resorb bone in vitro, and thus called DDOC. Because HA-coated plates, synthetic bone discs (FIG. 1G) and dentine slices all produced comparable results, HA-coated plates from a commercial source (OCT, CA) were utilized in subsequent experiments. In addition, both TRAP and resorptive activities were significantly higher than negative controls regardless of DC:T-cells ratio used (from 1:1 to 1:10). It is noteworthy that: i) there was no difference regarding DC and T-cells viability (see Table I below), and ii) no significant cell death was detected based on propidium iodide (PI) exclusion by FACS, suggesting that the phenotypic and functional changes on CD11c⁺DC are not associated with potential phagocytosis of apoptotic cells in the co-cultures. Furthermore, actin-ring formation characteristic of OC activity was detected in DDOC via Phalloidin staining (FIG. 1H). Interestingly, parallel analyses of the MACS-purified “splenic” CD11c⁺DC, showed that they behaved similarly (FIG. 1I), supporting the notion that the OC differentiation potential studied here is applicable to other CD11c⁺DC, not just those derived from BM cultures containing GM-CSF.

TABLE I
Viability of CD11c⁺ DCs in Co-cultures via Trypan-Blue Exclusion Analysis

| Time | DC only | DC + Aa | DC + T + Aa | DC + RANKL + Aa | DC + T + A + OPG |
|---|---|---|---|---|---|
| Day 5 | 60% + 0.1 | 97% + 0.2 | 95% + 0.25 (1:1) | 94% + 0.31 | 82% + 0.07 |
| | | | 74% + 0.02 (1:5) | | |
| | | | 80% + 0.25 (1:10) | | |
| | | | *[70-80%] | | |

*[Indicates the percentage of T-cell viability]

To investigate whether the development of DDOC is RANKL-dependent, OPG, anti-RANK Ab or isotype control was added into the co-cultures (Kong et al., “OPGL is a Key Regulator of Osteoclastogenesis, Lymphocyte Development and Lymph-Node Organogenesis,” Nature 397:315-323 (1999); Teng et al, “Functional Human T-Cell Immunity and Osteoprotegerin-Ligand (OPG-L) Control Alveolar Bone Destruction in Periodontal Infection,” J. Clin. Invest. 106:R59-R67 (2000), each of which is hereby incorporate by reference in its entirety). As expected, adding OPG and anti-RANK Ab completely abolished TRAP (p=0.01 & p=0.01, respectively: FIGS. 1B & 1G) and resorptive activities (p=0.02 & p=0.007, respectively: FIG. 1E & FIG. 1G); meanwhile, isotype-matched control Ab did not affect TRAP or resorptive activity (p>0.05), suggesting that RANKL/RANK signaling is required. To further confirm the contribution of RANKL in this process, 30 ng/ml of sRANKL (10 ng/ml-30 μg/ml titration tested) was added to CD11c⁺DC with or without Aa in the absence of CD4⁺ T-cells. Interestingly, sRANKL significantly restored TRAP (p=0.008; FIGS. 1B & 1G) and resorptive activities (p=0.01; FIGS. 1E & 1G) only in the presence of Aa, when compared to DC+RANKL. Therefore, both RANKL-RANK signaling and proper Ag stimulation allowed CD11c⁺DC to develop TRAP and resorptive activities. Since sRANKL (with Aa) restored about 75% of TRAP and 57% resorptive activities of those developed in DC+T+Aa co-cultures (with p=0.5; FIGS. 1A-1B & 1D-1E), respectively, contributions by T-cell signals or molecules other than RANKL cannot be totally excluded. Thus, it was concluded that depending on the signals encountered, CD11C⁺DC has the potential to develop OC phenotype and function in response to RANKL when stimulated in the bone environment in vitro.

Co-culturing CD11c⁺DC with T-cells and ConA resulted in significant TRAP and resorptive activity. Thus, to further confirm the role of T cell activation in the development of OC activity (i.e. TRAP and resorptive pits) by DC, the effects of anti-MHC-II (I-A^{d}) or -CD3/TCR blocking Abs were tested in the present co-cultures. Results showed that both Abs were able to abolish TRAP (p=0.008 & p=0.02; FIG. 1C) as well as resorptive activities (p=0.009 & p=0.01; FIG. 1F). Collectively, these data suggest that T-cell activation is involved in the development of TRAP and resorptive activities by CD11c⁺DC via RANKL production and possibly other molecules. Note that the total surface area and the total number of TRAP(+) cells gave comparable results; the total surface area was selected to represent TRAP activity in the examples below.

### Example 2

### OC Development from CD11c⁺DC is not Aa Specific or Restricted to Mo-Derived DC

To assess whether this phenomenon is Aa-specific or not, CD11c⁺DC were co-cultured with primed CD4⁺ T-cells (from Balb/c mice i.p. immunized with BI or OMP-1: 50 μg/ml in CFA 1:1 ratio) (Teng et al., “Evidence for Th2 Cell-Mediated Suppression of Antibody Response in Transgenic Beef Insulin-Tolerant Mice,” Eur J Immunol. 25: 2522-2527 (1995); Teng et al., “Expression Cloning of Periodontitis-Associated Apoptotic Factor, Cag-E Homologue, in Actinobacillus actinomycetemcomitans,” Biochem. Biophys. Res Communs. 303:1086-1094 (2003), each of which is hereby incorporated by reference in its entirety) and protein antigens BI or OMP-1 (20 μg/ml). The results showed that although TRAP appears to be slightly enhanced in OMP-1 and BI co-cultures compared to Aa (FIG. 2A), both TRAP and resorptive activities were comparable to those detected in DC+T-cells+Aa co-cultures (FIG. 2A), suggesting that this process is neither unique to Aa nor to microbial antigens. Moreover, when sonicated antigens of P. gingivalis (Pg) (Teng et al, “Functional Human T-Cell Immunity and Osteoprotegerin-Ligand (OPG-L) Control Alveolar Bone Destruction in Periodontal Infection,” J. Clin. Invest. 106:R59-R67 (2000), which is hereby incorporated by reference in its entirety) and E. coli were used, they induced TRAP and resorptive activities comparable to those induced by Aa in this co-culture system.

Any possible presence of Mo/MQ contaminants was next examined to further validate the findings described above. By FACS, it was confirmed that CD11C⁺DC are CD11b⁽⁻⁾F4/80⁽⁻⁾ER-MP12⁽⁻⁾ER-MP20⁽⁻⁾ (note that ER-MP12=CD31 and ER-MP20=Ly-6C; FIG. 2B), excluding the presence of: i) Mo/MQ contaminants, and ii) the classical CD11b⁺ OC precursors (Teitelbaum et al., “Genetic Regulation of Osteoclast Development & Function,” Nat. Rev. Genet. 8:638-649 (2003), which is hereby incorporated by reference in its entirety) in MACS-purified CD11c⁺DC. With that in mind, an assessment was made of the contribution of Mo/MQ to the generation of CD11C⁺DC. Therefore, mature Mo/MQ were depleted from BM cells before 7-day cultures followed by CD11c⁺DC purification by using anti-F4/80, CD11b, ER-MP12 and ER-MP20 mAbs and complements (Teng et al., “Evidence for Th2 Cell-Mediated Suppression of Antibody Response in Transgenic Beef Insulin-Tolerant Mice,” Eur J Immunol. 25:2522-2527 (1995); Leenen et al., “Murine Macrophage Precursor Characterization II. Monoclonal Antibodies Against Macrophage Precursor Antigens,” Eur. J. Immunol. 20:27-34 (1990), each of which is hereby incorporate by reference in its entirety). As expected, CD11c⁺DC derived from Mo/MQ-depleted or non-depleted BM cultures expressed similar levels of TRAP and resorptive activities by day 5 (FIGS. 2C & 2D), suggesting that CD11c⁺DC, regardless whether they were Mo/MQ-derived or not, are capable of developing OC activity. From these results it was concluded that CD11c⁺DC are indeed capable of developing into functional OC regardless of their source, i.e., whether they are derived from Mo/MQ or not. Furthermore, the frequency of TRAP(+) bone resorbing DDOC was the same whether DC were generated from Mo/MQ depleted or non-depleted BM. Despite that the complete exclusion of early OC progenitors is technically unfeasible due to the lack of specific markers, it is unlikely that the purified CD11c⁺DC contain DC/OC progenitors or early Mo precursors, because: i) addition of IL-4 & GM-CSF suppress Mo/MQ and promotes DC development in BM cultures, respectively (Schuler, “Chapter 27: A Guide to the Isolation and Propagation of Dendritic Cells,” In Dendritic Cells: Biology and Clinical Applications, Lotze et al. (eds.), San Diego, Calif., Academic, pp. 515-533 (1999), which is hereby incorporated by reference in its entirety), and ii) CD11c is a bona fide DC marker, not expressed by the majority of Mo/MQ or early DC/OC progenitors (Metlay et al., “The Distinct Leukocyte Integrins of Mouse Spleen Dendritic Cells as Identified with New Hamster Monoclonal Antibodies,” J. Exp. Med. 171:1753-1771 (1990); Nikolic et al., “Developmental Stages of Myeloid Dendritic Cells in Mouse Bone Marrow,” Int. Immunol. 15:515-524 (2003), each of which is hereby incorporate by reference in its entirety).

### Example 3

### Activated CD11c⁺DC Express Functional OC Phenotype

Freshly isolated CD11c⁺DC are bona fide DC, because they efficiently induced: i) proliferation of allogeneic CD4⁺ T-cells from C57BL/6 (H-2^{b}) in a mixed lymphocytes culture (FIG. 3A), and ii) activation of naïve syngeneic CD4⁺T-cells with Aa-antigens as determined by CD25 & RANKL expressions (see FIG. 3A), as confirmed by ELISA detection of RANKL in culture supernatants. CD11c⁺DC expressed no detectable CD80/CD86 and low cell surface MHC-II (sMHC-II: 1-15%) with majority of MHC-II localized intra-cellularly (iMHC-II=40-60%; day 0 of FIGS. 3B & 3B top-table), indicating immature phenotype. Importantly, they quickly up-regulated surface MHC-II and co-stimulatory molecules (CD80 & CD86) when activated by sonicated Aa-antigens and E. coli-LPS (FIG. 3B-top table).

The results of TRAP and resorptive pit assays prompted the characterization of DDOC phenotype. Since CT-R signifies OC function, anti-CT-R antibody was used to detect CT-R expression on DDOC, which has been shown to resemble CT-R detection by using radiolabelled CT-R (Quinn et al., “Calcitonin Receptor Antibodies in the Identification of Osteoclasts,” Bone. 25:1-8 (1999), which is hereby incorporated by reference in its entirety). The expression of other OC and DC surface markers on these cells was also assessed. Based on FACS results, the following results were obtained: i) on day O, CD11c⁺DC were CD11b⁽⁻⁾ F4/80⁽⁻⁾ Ly-6C⁽⁻⁾ CD3⁽⁻⁾, with a phenotype of GM-CSFR⁽⁺⁾RANK⁽⁺⁾MHC-II^{(low)} CT-R⁽⁻⁾ cathepsin-k⁽⁻⁾ (FIGS. 2B & 3B); ii) by day 3, significant percentage of CD11c⁺ cells up-regulated the expression of CT-R (<1%→58%), in contrast, GM-CSFR expression was down regulated (93%→21%). This change continued up to day 5 where CT-R expression peaked (93-96%) and GM-CSFR expression levelled off (≈25%; FIG. 3B). Thus, during activation and RANKL-RANK signaling, CD11c⁺DC developed into multinucleated TRAP(+), CT-R(+), cathepsin-k(+) OC with resorptive activity and an overall expression profile on days 3 & 5 as follows: CD11C⁽⁺⁾GMCSF-R^{(low to -)} RANK⁽⁺⁾MHC-II^{(hi)}CT-R⁽⁺⁾, cathepsin-k⁽⁺⁾, DEC205^{(low or -)} CD4⁽⁻⁾ CD8^{(+) or (-)} CD11b^{(low to -)} F4/80^{(+) or (-)} CD80^{(low to -)} CD86^{(low to -)} integrin αᵥ⁺β₃⁺ (FIG. 3B). CT-R & cathepsin-k expression by DDOC is consistent with bona fide OC phenotype (Teitelbaum et al., “Genetic Regulation of Osteoclast Development & Function,” Nat. Rev. Genet. 8:638-649 (2003), which is hereby incorporated by reference in its entirety). Importantly, OPG addition at the onset of the co-culture, significantly abolished CT-R expression (<10% by day 5; FIG. 3B), which correlated with the significantly reduced TRAP and resorptive activities detected (FIGS. 1B, 1E, 1G). These data clearly illustrate the OC development from activated CD11c⁺DC and further confirm the role of RANKL-RANK/OPG in this process. Furthermore, analysis of CT-R⁽⁺⁾DDOC revealed that the majority were CD11c⁺RANK⁺MHC-II⁺ GM-CSFR^{(low or -)} (FIG. 3C). Note that a relatively high MHC-II expression by DDOC is interesting and distinct from the classical OC phenotype (Teitelbaum et al., “Genetic Regulation of Osteoclast Development & Function,” Nat. Rev. Genet. 8:638-649 (2003), which is hereby incorporated by reference in its entirety). Whether this unique feature is associated with maintaining their APC function to prime CD4⁺ T-cells requires further study. On the other hand, GM-CSFR levels were down regulated regardless of OPG treatment, suggesting that RANKL-RANK signaling may not influence GM-CSFR expression.

To evaluate these phenotypic changes at the single cell level, CD11c⁺DC were CFSE-labelled prior to their co-culture. Cells were immuno-stained at different time points. Similar to FACS results, SIDIA showed that CT-R expression was significantly up-regulated and GM-CSFR down-regulated, while RANK levels remained relatively unchanged by day 3-5 of the co-culture. The addition of OPG significantly inhibited CT-R up-regulation associated with OC phenotype and function shown above (FIG. 3D). Together, these results strongly suggest that CD11c⁺DC can develop into functional OC after receiving appropriate activation signals in a bone environment (i.e. Ag & RANKL) and during interaction with activated CD4⁺T-cells.

### Example 4

### M-CSF is Essential for the Acquisition of CD11c⁺DC Potential to Act Like OC Precursors

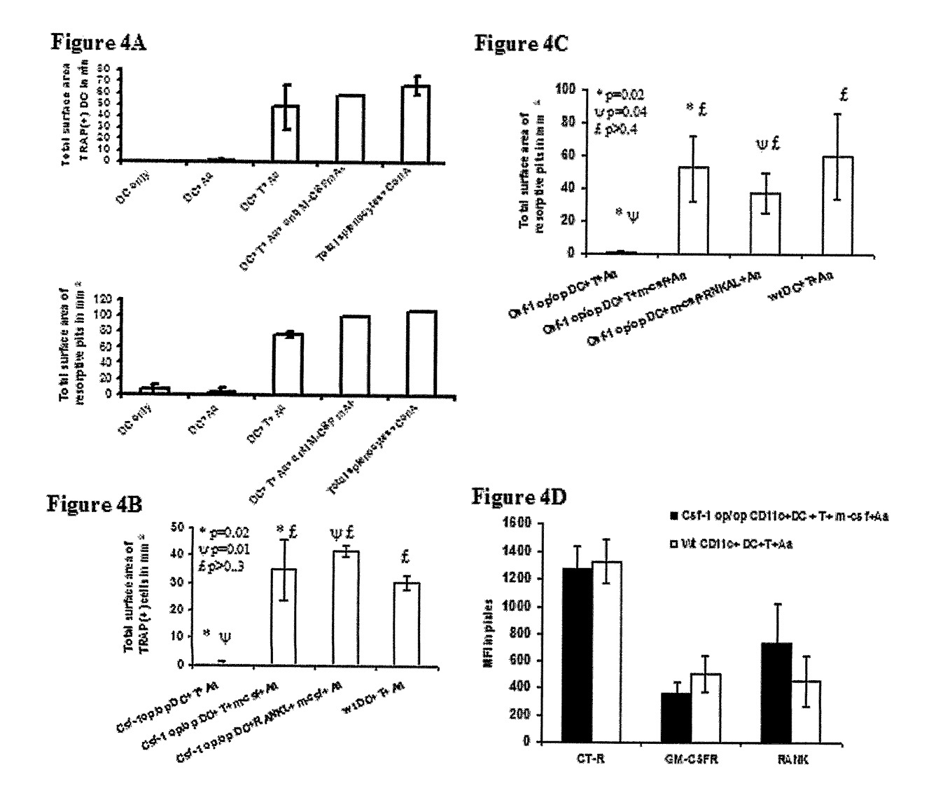

M-CSF is essential for the development of OC precursors as Csf-1^{-/-} op/op mice manifest severe osteopetrosis (Biskobing et al., “Characterization of M-CSF-Induced Proliferation and Subsequent Osteoclast Formation in Murine Marrow Culture,” J. Bone Miner. Res. 10: 1025-1032 (1995); Tanaka et al., “Macrophage Colony Stimulating Factor is Indispensable for Both Proliferation and Differentiation of Osteoclast Progenitors,” J. Clin. Invest. 91:257-263 (1993); Yoshida et al., “The Murine Mutation Osteopetrosis is in the Coding Region of the Macrophage Colony Stimulating Factor Gene,” Nature 345:442-444 (1990); Kodama et al., “Congenital Osteoclast Deficiency in Osteopetrotic (op/op) Mice is Cured by Injections of Macrophage-Colony Stimulating Factor,” J. Exp. Med. 173:269-272 (1991), each of which is hereby incorporate by reference in its entirety). Moreover, M-CSF is expressed by activated CD4⁺ T-cells (Weitzmann et al., “T Cell Activation Induces Human Osteoclast Formation Via Receptor Activator of Nuclear Factor Kappa B Ligand-Dependent and Independent Mechanisms,” JBMR 16:328-337 (2001), which is hereby incorporated by reference in its entirety) and was detected in the DC+T+Aa co-culture supernatants. Weitzman et al previously showed that osteoclastogenesis is independent of M-CSF produced by activated T-cells in vitro (Weitzmann et al., “T Cell Activation Induces Human Osteoclast Formation Via Receptor Activator of Nuclear Factor Kappa B Ligand-Dependent and Independent Mechanisms,” JBMR 16:328-337 (2001), which is hereby incorporated by reference in its entirety). To address whether M-CSF is involved in the process of DDOC development, 10 μg/ml anti-M-CSF Ab was added at the onset of co-culture. The results showed that there was no significant change in either TRAP or resorptive activity compared to that of DC+T+Aa (p>0.5; FIG. 4A), suggesting that M-CSF may not be directly involved in the development of DDOC in the above-described co-cultures.

Further investigation was performed to assess whether CD11c⁺DC generated from BM of osteopetrotic (Csf-1^{-/-}op/op) mice could give rise to TRAP and resorptive activities compared to those seen in Balb/c and genetically matched WT mice. To do so, CD11c⁺DC were purified from GM-CSF and IL-4 treated BM cultures of Csf-1^{-/-}op/op mice as described above. Due to the very low yield, BM cells from at least 5-7 op/op mice were pooled to generate sufficient numbers of CD11c⁺DC, which were then co-cultured with WT CD4⁺ T-cells and Aa-antigens with or without (25 ng/ml) rmM-CSF. The results showed that Csf-1^{-/-}op/op CD11c⁺DC did not develop TRAP or resorptive pit activity, when compared to WT (FIGS. 4B-4C). The addition of optimal rM-CSF (titration from 25 ng-25 μg/ml tested), significantly restored both TRAP and resorptive activities (FIGS. 4B-4C). Furthermore, replacement of CD4⁺T-cells with sRANKL yielded TRAP and resorptive activities comparable to those seen in the presence of T-cells, only when rM-CSF was added into the co-cultures (FIGS. 4B-4C). In parallel, the results of SIDIA at the single cell level further supported the above conclusion, as shown by an expression profile similar to those of WT DDOC (FIG. 4D).

To confirm the contribution of M-CSF during CD11c⁺DC's development into OC, WT CD11c⁺DC were co-cultured with Csf-1^{-/-}op/op-derived CD4⁺T-cells and Aa. Interestingly, WT CD11c⁺DC were capable of developing into TRAP(+) functional OC under these conditions. Csf-1^{-/-} op/op CD11c⁺DC on the other hand did not develop TRAP or resorptive pits unless exogenous M-CSF was added during their co-culture with T-cells or sRANKL and Aa (FIG. 4B-4C). Collectively, these data (FIG. 4A-4C) suggest that the exposure to M-CSF during DC development is necessary for the acquisition of their osteoclastogenic potential and that M-CSF is not required for the development of WT CD11c⁺DC into OC in this co-culture system. These findings are consistent with the current understanding that RANKL is essential for the differentiation and activation of bona fide OC, whereas M-CSF is required for the development and survival of OC precursors (Theill et al., “T cell, Bone Loss and Mammalian Evolution,” Annu. Rev. Immunol. 20:795-823 (2002); Lacey et al., “Osteoprotegerin Ligand is a Cytokine that Regulates Osteoclast Differentiation and Activation,” Cell 93:165-176 (1998); Yasuda et al., “Osteoclast Differentiation Factor is a Ligand for Osteoprotegerin/Osteoclastogenesis-Inhibitory Factor and is Identical to TRANCE/RANKL,” Proc. Natl. Acad. Sci. USA 95:3597-3602 (1998); Teitelbaum et al., “Genetic Regulation of Osteoclast Development & Function,” Nat. Rev. Genet. 8:638-649 (2003); Biskobing et al., “Characterization of M-CSF-Induced Proliferation and Subsequent Osteoclast Formation in Murine Marrow Culture,” J. Bone Miner. Res. 10: 1025-1032 (1995); Yoshida et al., “The Murine Mutation Osteopetrosis is in the Coding Region of the Macrophage Colony Stimulating Factor Gene,” Nature 345:442-444 (1990); Mizuno et al., “Severe Osteoporosis in Mice Lacking Osteoclastogenesis Inhibitory Factor/Osteoprotegerin,” Biochem. Biophys. Res. Commun. 247:610-615 (1998), each of which is hereby incorporate by reference in its entirety).

### Example 5

### CD11c⁺DC-Derived OC can Induce Bone Resorption In Vivo

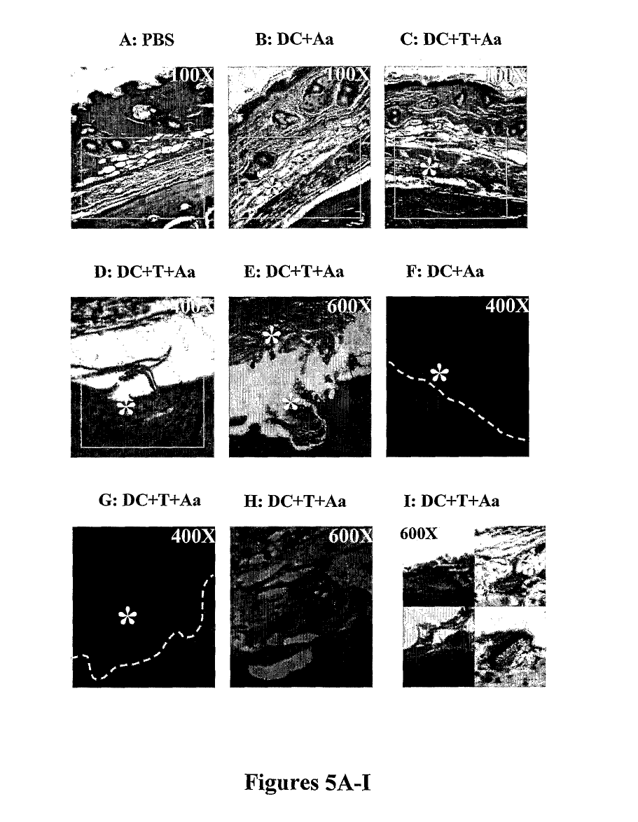

To assess whether or not DDOC can induce bone resorption in vivo, not just in vitro, CFSE-labelled CD11c⁺DC were co-cultured in vitro as described above for 2.5 to 3 days before injection onto the periostium of NOD/SCID mice calvaria. This protocol allowed the in situ detection of labelled cells post-injection. The results of histological and immunohistochemical analyses revealed that injection of PBS-only, CD4⁺T-cells alone or CD11c⁺DC alone (Aa stimulated or not) did not induce bone resorption in vivo (FIGS. 5A, B & F). In contrast, CFSE-labelled CD11c⁺DC from DC+T+Aa (i.e. DDOC) were TRAP(+) post-adoptive transfer (FIGS. 5C, 5D, 5E, 5G, 5I) where they: i) induced significantly higher bone resorption than the control groups (as evidenced by the eroded bone surfaces highlighted on the histological sections of FIG. 5F-5G) and ii) maintained CD11c expression in vivo. These findings suggest that, after proper stimulation, CD11c⁺DC can develop into activated OC capable of resorbing bone in vivo.

### Discussion of Examples 1-5

DC are a heterogeneous population of leukocytes whose immune functions have been extensively studied although their origin and developmental pathways remain inconclusive (Banchereau et al., “Immunobiology of Dendritic Cells,” Annu. Rev Immunol. 18:767-811 (2000); Ardavin et al., “Dendritic Cell: Immunobiology & Cancer Immunotherapy,” Immunity 1:17-23 (2004); Ardavin, “Origin, Precursors and Differentiation of Mouse Dendritic Cells,” Nat Rev. Immunol. 3:582-590 (2003); Martinez del Hoyo et al., “Dendritic Cell Differentiation Potential of Mouse Monocytes: Monocytes Represent Immediate Precursors of CD8⁻ and CD8⁺ Splenic Dendritic Cells,” Blood 103:2668-2676 (2004); Traver et al., “Development of CD8a-Positive Dendritic Cells From a Common Myeloid Progenitor,” Science 290:2152-2154 (2000); Pullendran et al., “Sensing Pathogens and Tuning Immune Responses,” Science 293:253-256 (2001); Shortman, “Burnet Oration: Dendritic Cells: Multiple Subtypes, Multiple Origins, Multiple Functions,” Immunol. Cell Biol. 78:161-165 (2000), each of which is hereby incorporate by reference in its entirety). It is evident that i) CD11c⁺DC are present in the inflamed synovia of rheumatoid arthritis (Thomas et al., “Dendritic Cells and the Pathogenesis of Rheumatoid Arthritis,” J. Leukoc. Biol. 66:286-92 (1999); Highton et al., “Cells Expressing Dendritic Cell Markers are Present in the Rheumatoid Nodule,” J. Rheumatol. 27:339-346 (2000), each of which is hereby incorporate by reference in its entirety) and ii) DC/T-cell interactions play a pivotal role in inflammatory bone disorders like rheumatoid arthritis and periodontal disease (Thomas et al., “Dendritic Cells and the Pathogenesis of Rheumatoid Arthritis,” J. Leukoc. Biol. 66:286-92 (1999); Highton et al., “Cells Expressing Dendritic Cell Markers are Present in the Rheumatoid Nodule,” J. Rheumatol. 27:339-346 (2000); Cirrincione et al., “Lamina Propria Dendritic Cells Express Activation Markers and Contact Lymphocytes in Chronic Periodontitis,” J. Periodontol. 73:45-52 (2002); Teng. “The Role of Acquired Immunity and Periodontal Disease Progression,” Crit Rev Oral Biol Med. 14:237-252 (2003); Santiago-Schwarz et al., “Dendritic Cells (DCs) in Rheumatoid Arthritis (RA): Progenitor Cells and Soluble Factors Contained in RA Synovial Fluid Yield a Subset of Myeloid DCs that Preferentially Activate Th1 Inflammatory-Type Responses,” J. Immunol. 67:1758-1768 (2001), each of which are hereby incorporated by reference in its entirety) and possibly osteomyelitits.

Exploring DC functions associated with the inflammation-induced bone loss will aid a better understanding of its mechanisms and potential therapeutic strategies for inflammatory bone disorders. The above findings indicate a potentially significant contribution of DC, at least CD11c⁺DC subset(s), to elevated osteoclastogenesis associated with inflammatory bone disorders, where they may not only act as potent APC for the activation and regulation of adaptive immunity, but also as OC precursors, directly involved in bone loss. Results from M-CSF blocking (FIG. 4A), co-culturing WT DC with csf-1^{-/-}op/op-derived T-cells and Aa, and M-CSF rescue studies (FIG. 4B-D) indicate that M-CSF is not required for OC development from WT CD11c⁺DC in the co-culture system and suggest a stage-specific requirement of M-CSF “before” their transition to OC up-stream of RANKL-RANK signaling. Exposure of OC precursors to M-CSF has been shown to be essential for the development of their osteoclastogenic potential possibly by up-regulating RANK expression (Theill et al., “T cell, Bone Loss and Mammalian Evolution,” Annu. Rev. Immunol. 20:795-823 (2002); Biskobing et al., “Characterization of M-CSF-Induced Proliferation and Subsequent Osteoclast Formation in Murine Marrow Culture,” J. Bone Miner. Res. 10:1025-1032 (1995), each of which is hereby incorporated by reference in its entirety). Alternatively, one may suspect the presence of few contaminating precursors among WT DC that are absent in Csf-1^{-/-}op/op DC cultures. The CD11c⁺DC subset studied here was highly pure, because: i) it has been shown that CD11c is not expressed on early precursors (Nikolic et al., “Developmental Stages of Myeloid Dendritic Cells in Mouse Bone Marrow,” Int. Immunol. 15:515-524 (2003), which is hereby incorporated by reference in its entirety) and ii) the presence of both MQ (CD11b⁺ & F4/80⁺) and Mo (ER-MP12⁺ & ER-MP20⁺) contaminants were excluded (FIG. 2A). Thus, this alternative possibility is unlikely.

In contrast to the proposed model by Miyamoto et al., where committed DC derived from the common DC/OC progenitors lack the ability to develop into OC (Miyamoto et al., “Bifurcation of Osteoclasts and Dendritic Cells From Common Progenitors,” Blood 98:2544-2554 (2001), which is hereby incorporated by reference in its entirety), the above data demonstrate CD11c⁺DC development into functional multinucleated TRAP⁺ CT-R⁺ cathepsin-k⁺ MHC-II⁺ OC, whereby they acquire the ability to induce bone resorption not only in vitro but also in vivo (FIGS. 1 & 5). This suggests that they posses certain developmental plasticity and may represent an alternative OC differentiation pathway. The molecular mechanisms responsible for such development appear to involve primarily RANKL/RANK signaling in addition to antigen activation possibly via toll-like receptors. Various effects of LPS on osteoclastogenesis in the presence or absence of OB/stromal cells have been reported, and its precise role remains undetermined (Abu-Amer et al., “Lipopolysaccharide-Stimulated Osteoclastogenesis is Mediated by Tumor Necrosis Factor Via its P55 Receptor,” J. Clin. Invest. 100:1557-1565 (1997); Kikuchi et al., “Gene Expression of Osteoclast Differentiation Factor is Induced by Lipopolysaccharide in Mouse Osteoblasts Via Toll-Like Receptors,” J Immunol. 166:3574-3579 (2001); Jiang et al., “Bacteria Induce Osteoclastogenesis Via an Osteoblast-Independent Pathway,” Infect Immun. 70(6):3143-3148 (2002), each of which is hereby incorporated by reference in its entirety). By using Mo from BM, it was recently shown that in the absence of OC or stromal cells, direct LPS interaction with OC precursors could result in inhibition or enhancement of osteoclastogenesis depending on their differentiation stage (Zou et al., “Dual Modulation of Osteoclast Differentiation by Lipopolysaccharide,” J Bone Miner Res. 17(7):1211-8 (2002), which is hereby incorporated by reference in its entirety). In the present study, Aa-antigens & OMP-1 (with traces of LPS) and LPS-free BI protein all yield comparable TRAP & resorptive activities (FIG. 2A). Secondly, there was no detectable TRAP or resorptive activities in CD11c⁺DC when co-cultured with E. coli-derived LPS in the presence or absence of sRANKL. These findings argue against a direct role for LPS (at least TLR-4) in OC development in this system, in agreement with the report by Takami et al., where LPS directly inhibits osteoclastogenesis from OC precursors (Takami et al., “Stimulation by Toll-Like Receptors Inhibits Osteoclast Differentiation,” J Immunol. 169:1516-1523 (2002), which is hereby incorporated by reference in its entirety).

The cellular signaling pathways of RANKL/RANK in OC and/or OC precursors involve tumor necrosis factor receptor-associated factors (TRAFs), p38 MAPK, ERK, JNK and downstream transcription factors such as NF-κB, and c-Fos associated with AP-1 (Blair et al., “Osteoclast Signaling Pathways,” Biochem. Biophys. Res. Comm. 328:728-738 (2005), which is hereby incorporated by reference in its entirety); however, it remains unclear whether it is the case for CD11c⁺DC. Moreover, multiple immunoglobulin like receptors associated with immunoreceptor tyrosine-based activation motif (ITAM)-harboring adaptors, Fc receptor common 7 chain, and DNAX-activating protein (DAP-12) have been recently implicated in osteoclastogenesis, which further strengthens the link between the immune and bone remodeling (Koga et al., “Costimulatory Signals Mediated by the ITAM Motif Cooperate with RANKL for Bone Homeostasis,” Nature 428:758-763 (2004), which is hereby incorporated by reference in its entirety). Whether these factors play any role in the development of DDOC remains to be explored.

Rivollier et al. recently demonstrated that human circulating blood Mo-derived DC were capable of trans-differentiating into bone resorbing OC in the presence of M-CSF and RANKL in vitro (Rivollier et al., “Immature Dendritic Cell Transdifferentiation into Osteoclasts: A Novel Pathway Sustained by Rheumatoid Arthritis Microenvironment,” Blood 104:4029-4037 (2004), which is hereby incorporated by reference in its entirety). The data presented in the preceding examples clearly indicate that interactions of BM-derived and splenic CD11c⁺DC with CD4⁺T-cells in the bone environment can promote DDOC development in response to microbial or protein antigens and RANKL-RANK signaling (FIGS. 1A & 1D). Despite that in both studies DDOC develop under different conditions and kinetics, they appear to carry similar phenotypes with some differences (see Table II below: murine DDOC are CD86⁻CD11b⁻ and possess dendrites). Nevertheless, based on the results of both studies, it is reasonable to believe that DDOC may develop not only in the presence of M-CSF & RANKL produced by OB, synovia, and stromal cells (Takayanagi et al., “Involvement of Receptor Activator of Nuclear Factor KappaB Ligand/Osteoclast Differentiation Factor in Osteoclastogenesis From Synoviocytes in Rheumatoid Arthritis,” Arthritis Rheum. 43:259-69 (2000), which is hereby incorporated by reference in its entirety), but also in response to RANKL produced by inflammatory T-cells and stimulatory antigens during immune interactions in the bone environment.

TABLE II
Comparison Between Murine and Human DC-derived OC

| Criteria | Murine DC-Derived OC | Human DC-Derived OC* |
|---|---|---|
| Origin | BM-derived or splenic CD11c⁺ DC | Circulating blood Mo-derived DC |
| Differentiation | RANKL, Ag stimulation | M-CSF & RANKL |
| Signals | | |
| Differentiation | 2-2.5 days in vitro | 12 days in vitro |
| Kinetics | | |
| Morphology | Micronucleated; some with dendrites | Micronucleated; no dendrites |
| Phenotype | At day 5: | At day 12: |
| | TRAP⁺, CT-R⁺, Cathepsin K⁺, | TRAP⁺, CT-R⁺, Cathepsin K⁺, |
| | integrins αv⁺β3⁺, CD11b⁻ | integrins αv⁺β3⁺, CD11b⁺, CD11c⁺, |
| | (80%), CD11c⁺, MHC-II⁺, CD86⁻ | HLA-DR⁺, CD86⁺ |
| | (87%) also RANK⁺, GM-CSFR⁻ | |
| Bone Resorption | Yes | Yes |

*Rivollier et al., “Immature Dendritic Cell Transdifferentiation into Osteoclasts: A Novel Pathway Sustained by Rheumatoid Arthritis Microenvironment,” Blood 104:4029-4037 (2004).

In summary, the development of functional OC has been demonstrated using CD11c⁺DC subset(s), and the OC are capable of inducing bone resorption in vitro and in vivo. The dynamic process of OC differentiation, development and activation from CD11c⁺DC through orchestrated M-CSF and RANKL signaling (and overall activation signals) illustrates the complexity of DC biology. This DC plasticity supports a link between innate immunity and osteoclastogenesis, beyond the current paradigm of osteoimmunology, and furthers the understanding of the immune interactions involved in bone remodeling under pathological conditions such as rheumatoid arthritis, periodontal disease, osteomyelitis and other inflammatory bone disorders.

### Example 6

### Analysis of Histological Sections of Induced Arthritic Joints

Chicken type-II collagen in CFA was used to immunize and induce RA in the acceptable DBA mice according to the original protocol of Stuart et al. (J. Clin. Invest. 69:673-683 (1982), which is hereby incorporated by reference in its entirety. Control DBA mice received PBS only.

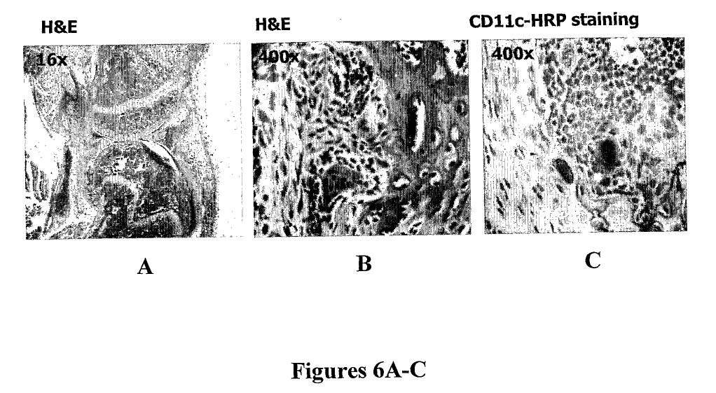

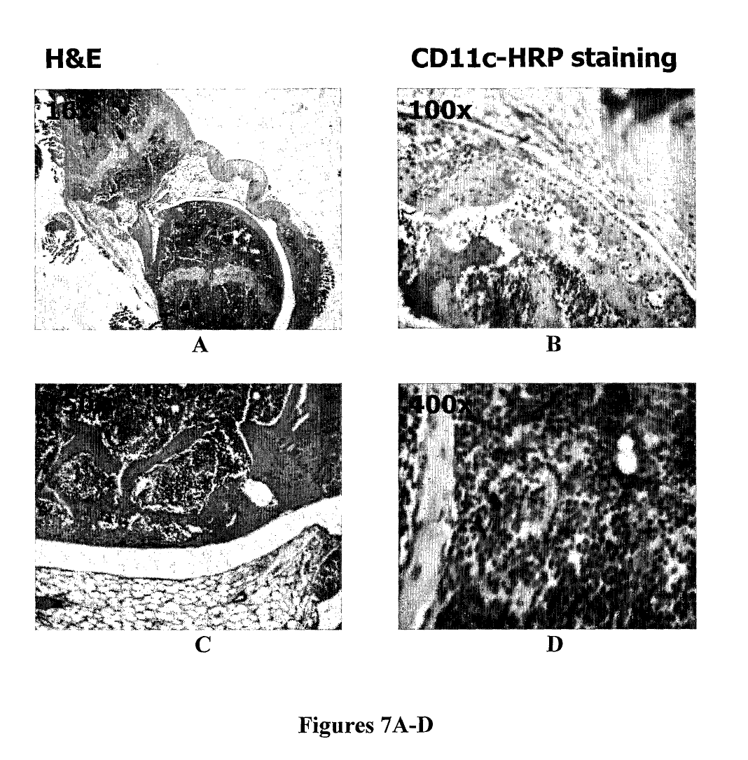

Lower power of H&E staining showing severe RA induced in DBA mice (FIG. 6A) while the PBS-injected mice showed normal knee joint architectures (FIG. 7A). The higher magnifications of H&E show active bone resorptive activity with multinucleated giant cell-like osteoclasts on eroded bone surfaces in mice with RA (FIG. 6B, arrow); while the normal control mice manifest no detected bone resorptive activity (no sign of OC activity/presence) (FIG. 7C). The immuno-histochemisty staining using anti-CD11c⁺ monoclonal antibody showed positive staining of the multinucleated giant-cell like osteoclasts in the RA mice (FIG. 6C, arrow). The normal control mice without RA showed no detectable CD11c⁺ cells in the joint (FIG. 7B), but only detectable in the bone marrow cells/space (serving as an internal positive control for the immuno-staining) (FIG. 7D, arrows).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An ex vivo method of producing osteoclasts, said method comprising:
providing isolated CD11c⁺ dendritic cells;
culturing the CD11c⁺ dendritic cells in culture medium under conditions effective to produce an osteoclast precursor population; and
treating the osteoclast precursor population under conditions effective to induce differentiation of the osteoclast precursor population to form osteoclasts.

2. The method according to claim 1, wherein the culture medium during said culturing comprises macrophage colony stimulating factor and IL-4.

3. The method according to claim 1, wherein said culturing is carried out from about two to about seven days.

4. The method according to claim 1 further comprising:
substantially purifying the osteoclast precursor population prior to said treating.

5. The method according to claim 4, wherein said substantially purifying is carried out using a cell sorter.

6. The method according to claim 1, wherein said treating comprises exposing the osteoclast precursor population to a peptide or polypeptide antigen, receptor activator of NF-κB ligand (RANKL), and bone or a bone substitute.

7. The method according to claim 6, wherein RANKL is introduced to the osteoclast precursor population.

8. The method according to claim 6, wherein T cells expressing RANKL are introduced to the osteoclast precursor population.

9. The method according to claim 6, wherein the peptide or polypeptide antigen is a microbial antigen.

10. The method according to claim 9, wherein the microbe is A. actinomycetemcomitans, P. gingivalis, or E. coli.

11. The method according to claim 6, wherein the bone or bone substitute is selected from the group consisting of bone, dentin, synthetic bone, hydroxyapatite, or combinations thereof

12. The method according to claim 1, further comprising isolating the CD11c⁺ dendritic cells from blood or tissue.

13. The method according to claim 11, wherein the CD11c⁺ dendritic cells are isolated from a tissue selected from the group consisting of bone marrow, peripheral blood, spleen, and lymph nodes.

14. The method according to claim 1, wherein the CD11c⁺ cells are mammalian cells.

15. The method according to claim 14, wherein the mammalian cells are human cells or rodent cells.

16. The method according to claim 1, further comprising identifying osteoclasts produced during said second culturing.

17. The method according to claim 16, wherein said identifying comprises determining which osteoclasts 1) express tartrate resistant acid phosphatase, 2) resorb bone, 3) have calcitonin receptors, or 4) combinations thereof.

18. Osteoclasts cultured from CD11c⁺ dendritic cells according to the method of claim 1.

19. The osteoclasts according to claim 18, wherein the osteoclasts are cd11c⁺, cd11b⁻.

20. The osteoclasts according to claim 18, wherein the osteoclasts are TRAP⁺, CT-R⁺.

21. A method of upregulating bone resorption in a subject comprising:
performing the method according to claim 1 to form osteoclasts using CD11c⁺ dendritic cells isolated from a subject; and
returning the osteoclasts to the subject, thereby upregulating bone resorption in the subject.

22. A method of upregulating bone resorption in a subject comprising:
providing CD11c⁺ dendritic cells isolated from a subject;
introducing a nucleic acid molecule to the CD11c⁺ dendritic cells to form transgenic CD11c⁺ dendritic cells, wherein the nucleic acid molecule encodes a protein or polypeptide capable of promoting osteoclastogenesis of the dendritic cells; and
returning the transgenic cells to the subject, thereby upregulating bone resorption in the subject.

23. The method according to claim 22, wherein the nucleic acid molecule introduced into the CD11c⁺ dendritic cells is in an expression vector, and wherein the nucleic acid molecule is inserted in the expression vector in a sense (5′→3′) orientation that allows expression of the protein or polypeptide by the dendritic cells.

24. The method according to claim 22, wherein the protein or polypeptide encoded by the nucleic acid molecule is a bone modulating peptide, protein, chemokine, cytokine, growth hormone, or monoclonal antibody.

25. The method according to claim 22 further comprising:
exposing the transgenic cells to stimuli to produce CD11c⁺ osteoclasts ex vivo.

26. The method according to claim 25, wherein said exposing comprises:
culturing the CD11c⁺ dendritic cells in culture medium under conditions effective to produce an osteoclast precursor population;
substantially purifying the osteoclast precursor population; and
treating the substantially pure osteoclast precursor population under conditions effective to induce differentiation of the osteoclast precursor population to form osteoclasts.

27. The method according to claim 26, wherein the culture medium during said culturing comprises macrophage colony stimulating factor and IL-4.

28. The method according to claim 26, wherein said treating comprises exposing the substantially pure osteoclast precursor population to a peptide or polypeptide antigen, receptor activator of NF-κB ligand (RANKL), and bone or a bone substitute.

29. The method according to claim 22, wherein the subject is a mammal.

30. The method according to claim 29, wherein the mammal is human.

31. A method of upregulating bone resorption in a subject comprising:
providing naïve CD4⁺ T cells isolated from the subject;
co-culturing the CD4⁺ T cells with a peptide or polypeptide antigen and CD11c⁺ dendritic cells under conditions effective to promote osteoclastogenesis of the CD11c⁺ dendritic cells; and
returning the cultured CD4⁺ T cells to the subject, where they are in contact with CD11c⁺ dendritic cells, thereby promoting osteoclastogenesis of the CD11c⁺ dendritic cells and upregulating bone resorption in the subject.

32. The method according to claim 31, wherein the protein antigen is a microbial antigen.

33. The method according to claim 32, wherein the microbe is A. actinomycetemcomitans or P. gingivalis

34. The method according to claim 31, wherein CD4⁺ T cells are isolated from blood or tissue.

35. The method according to claim 34, wherein the CD4⁺ T cells are isolated from a human.

36. The method according to claim 34, wherein the CD11c⁺ dendritic cells are isolated from a tissue of the subject selected from the group consisting of bone marrow, spleen, and lymph nodes.

37. The method according to claim 36 further comprising prior to said co-culturing:
culturing the CD11c⁺ dendritic cells in culture medium under conditions effective to produce an osteoclast precursor population; and
substantially purifying the osteoclast precursor population.

38. The method according to claim 31, wherein both the CD4⁺ T cells and the CD11c⁺ cells are returned.

39. A method of downregulating bone resorption in a subject comprising:
providing CD11c⁺ dendritic cells isolated from a subject;
introducing a nucleic acid molecule to the CD11c⁺ dendritic cells to form transgenic CD11c⁺ dendritic cells, wherein the nucleic acid molecule expresses a protein or polypeptide or an RNA molecule capable of inhibiting osteoclastogenesis in CD11c⁺ dendritic cells; and
returning the transgenic CD11c⁺ dendritic cells to the subject, thereby downregulating bone resorption in the subject.

40. The method according to claim 39, wherein the nucleic acid molecule introduced into the CD11c⁺ dendritic cells is in an expression vector, and wherein expression of the nucleic acid molecule in the CD11c⁺ dendritic cells effects downregulation of osteoclastogenesis in dendritic cells by a form of post-transcriptional gene silencing.

41. The method according to claim 40, wherein the form of post-transcriptional gene silencing is RNA interference.

42. The method according to claim 41, wherein the nucleic acid molecule is modified to encode a protein or polypeptide transcribed in the transgenic cell as double-stranded RNA.

43. The method according to claim 40, wherein the nucleic acid molecule is modified to encode an antisense nucleic acid complementary to a nucleic acid molecule encoding a protein or polypeptide capable of promoting osteoclastogenesis in CD11c⁺ dendritic cells.

44. The method according to claim 39, wherein the protein or polypeptide is a bone modulating peptide, protein, chemokine, cytokine, growth hormone, or monoclonal antibody.

45. The method according to claim 39, wherein the nucleic acid molecule encodes a protein or polypeptide selected from the group consisting of osteoprotegerin, GM-CSF, and anti-RANKL antibody.

46. The method according to claim 39, wherein the CD11c⁺ dendritic cells are isolated from a tissue selected from the group consisting of blood, bone marrow, spleen, and lymph nodes.

47. The method according to claim 39, wherein the subject is human.

48. A method of inhibiting osteoclastogenesis of CD11c⁺ dendritic cells in a subject comprising:
administering to a subject an effective amount of an anti-RANKL antibody, where said administering is effective to contact RANKL in the proximity of CD11c⁺ dendritic cells, thereby inhibiting RANKL-mediated osteoclastogenesis of the CD11c⁺ dendritic cells.

49. A method of downregulating bone resorption in a subject comprising:
providing naïve CD4⁺ T cells isolated from a subject;
introducing a nucleic acid molecule to the CD4⁺ T cells to form transgenic CD4⁺ T cells, wherein the nucleic acid molecule encodes a protein or polypeptide that inhibits osteoclastogenesis in CD11c⁺ dendritic cells, and
returning the transgenic CD4⁺ T cells to the subject, where the T cells inhibit osteoclastogenesis of CD11c⁺ dendritic cells, thereby down-regulating bone resorption in the subject.

50. The method according to claim 49, wherein the nucleic acid molecule introduced into the CD4⁺ T cells is in an expression vector.

51. The method according to claim 50, wherein the nucleic acid molecule is inserted into the expression vector in a sense orientation and is operably associated with 5′ and 3′ regulatory regions to allow expression of the protein or polypeptide.

52. The method according to claim 49, wherein the nucleic acid molecule encodes a protein or polypeptide that is a bone modulating peptide, protein, cytokine, growth hormone, or monoclonal antibody.

53. The method according to claim 52, wherein the nucleic acid molecule encodes a protein or polypeptide selected from the group consisting of osteoprotegerin, GM-CSF, and anti-RANKL antibody.

54. A method of treating a subject for a bone disease or disorder involving excessive bone deposition, said method comprising:
performing the method according to claim 21 on a subject having a bone disease or disorder involving excessive bone deposition, wherein the returned osteoclasts promote bone resorption and thereby treat the bone disease or disorder.

55. The method according to claim 54 wherein the bone disease or disorder is osteopetrosis or osteoarthritis.

56. A method of treating a subject for a bone disease or disorder involving excessive bone deposition, said method comprising:
performing the method according to claim 22 on a subject having a bone disease or disorder involving excessive bone deposition, wherein the returned transgenic cells promote osteoclastogenesis of CD11c⁺ dendritic cells and thereby treats the bone disease or disorder.

57. The method according to claim 56 wherein the bone disease or disorder is osteopetrosis or osteoarthritis.

58. A method of treating a subject for a bone disease or disorder involving excessive bone deposition, said method comprising:
performing the method according to claim 31 on a subject having a bone disease or disorder involving excessive bone deposition, wherein the returned CD4⁺ T cells promote osteoclastogenesis of CD11c⁺ dendritic cells and thereby treats the bone disease or disorder.

59. The method according to claim 58 wherein the bone disease or disorder is osteopetrosis or osteoarthritis.

60. A method of treating a subject for a bone disease or disorder involving excessive bone resorption, said method comprising:
performing the method according to claim 39 on a subject having a bone disease or disorder involving excessive bone resorption, wherein the returned transgenic CD11c⁺ dendritic cells are inhibited against osteoclastogenesis, thereby treating the bone disease or disorder.

61. The method according to claim 60 wherein the bone disease or disorder is osteoporosis, osteomyelitis, rheumatoid arthritis, periodontal disease, or Pagets Disease.

62. A method of treating a subject for a bone disease or disorder involving excessive bone resorption, said method comprising:
performing the method according to claim 48 on a subject having a bone disease or disorder involving excessive bone resorption, wherein the administering of the anti-RANKL antibody inhibits RANKL-mediated osteoclastogenesis of CD11c⁺ dendritic cells, thereby treating the bone disease or disorder.

63. The method according to claim 62 wherein the bone disease or disorder is osteoporosis, osteomyelitis, rheumatoid arthritis, periodontal disease, or Pagets Disease.

64. A method of treating a subject for a bone disease or disorder involving excessive bone resorption, said method comprising:
performing the method according to claim 49 on a subject having a bone disease or disorder involving excessive bone resorption, wherein the returned transgenic CD4⁺ T cells inhibited osteoclastogenesis of CD11c⁺ dendritic cells, thereby treating the bone disease or disorder.

65. The method according to claim 64 wherein the bone disease or disorder is osteoporosis, osteomyelitis, rheumatoid arthritis, periodontal disease, or Pagets Disease.

66. A method of screening for compounds which affect dendritic cell osteoclastogenesis comprising:
performing the method according to claim 1;
exposing the dendritic cell or osteoclast precursor to a compound; and
determining the ability of the compound of affect dendritic cell osteoclastogenesis.

67. The method according to claim 66, wherein the ability of a compound to affect osteoclastogenesis is determined by osteoclast cell count, TRAP staining, bone resorption assay, or a combination thereof.

68. The method according to claim 66, wherein the affect of the compound is to stimulate osteoclastogenesis.

69. The method according to claim 66, wherein the affect of the compound is to inhibit osteoclastogenesis.

70. A method of identifying genes related to the production of functional osteoclasts from CD11c⁺ dendritic cells, said method comprising:
performing the method according to claim 1 while altering one or more conditions;
determining osteoclast production and function in the culture; and
screening for one or more genes associated with a change in osteoclast production or function in the culture compared with a culture in which culture conditions were not altered.

71. The method according to claim 70, wherein said determining osteoclast production is carried out by measuring osteoclast cell count, TRAP staining, bone resorption, or a combination thereof.

72. The method according to claim 70, wherein said screening is carried out by differential gene display, RNA arbitrarily primed (RAP)-PCR, or gene microarray analysis.
